(19) United States
(12) Patent Application Publication
(10) Pub. No.: US 2002/0127537 A1
(43) Pub. Date: Sep. 12, 2002

(54) Herbicide target genes and methods

(76) Inventors: David A. Patton, Basel (CH); Carl S. Ashby, Glen Allen, VA (US); Sandra L. Volrath, Durham, NC (US); John A. McElver, Research Triangle Park, NC (US); Michael W. Bauer, Research Triangle Park, NC (US)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 09/893,817

(22) Filed: Jun. 28, 2001

Related U.S. Application Data
(60) Provisional application No. 60/214,819, filed on Jun. 28, 2000.

(51) Int. Cl. C12Q 1/00; A01N 25/00; C12N 5/04

(52) U.S. Cl. 435/4; 435/410; 504/116.1

(57) ABSTRACT

The invention relates to genes isolated from Arabidopsis that code for proteins essential for normal plant development. The invention also includes the methods of using these proteins to discover new herbicides, based on the essentiality of the genes for normal growth and development. The invention can also be used in a screening assay to identify inhibitors that are potential herbicides. The invention is also applied to the development of herbicide tolerant plants, plant tissues, plant seeds, and plant cells.

This application claims the benefit of U.S. Provisional Application No. 60/214,819, filed Jun. 28, 2000, incorporated herein by reference in its entirety.

### FIELD OF THE INVENTION

The invention relates to genes isolated from Arabidopsis thaliana that encode proteins essential for plant growth and development. The invention also includes the methods of using these proteins as herbicide targets, based on the essentiality of these genes for normal growth and development. The invention is also useful as a screening assay to identify inhibitors that are potential herbicides. The invention may also be applied to the development of herbicide tolerant plants, plant tissues, plant seeds, and plant cells.

### BACKGROUND OF THE INVENTION

The use of herbicides to control undesirable vegetation such as weeds in crop fields has become almost a universal practice. The herbicide market exceeds 15 billion dollars annually. Despite this extensive use, weed control remains a significant and costly problem for farmers.

Effective use of herbicides requires sound management. For instance, the time and method of application and stage of weed plant development are critical to getting good weed control with herbicides. Since various weed species are resistant to herbicides, the production of effective new herbicides becomes increasingly important. Novel herbicides can now be discovered using high-throughput screens that implement recombinant DNA technology. Metabolic enzymes found to be essential to plant growth and development can be recombinantly produced through standard molecular biological techniques and utilized as herbicide targets in screens for novel inhibitors of the enzyme activity. The novel inhibitors discovered through such screens may then be used as herbicides to control undesirable vegetation.

Herbicides that exhibit greater potency, broader weed spectrum, and more rapid degradation in soil can also, unfortunately, have greater crop phytotoxicity. One solution applied to this problem has been to develop crops that are resistant or tolerant to herbicides. Crop hybrids or varieties tolerant to the herbicides allow for the use of the herbicides to kill weeds without attendant risk of damage to the crop. Development of tolerance can allow application of a herbicide to a crop where its use was previously precluded or limited (e.g. to pre-emergence use) due to sensitivity of the crop to the herbicide. For example, U.S. Pat. No. 4,761,373 to Anderson et al. is directed to plants resistant to various imidazolinone or sulfonamide herbicides. This resistance is conferred by an altered acetohydroxyacid synthase (AHAS) enzyme. U.S. Pat. No. 4,975,374 to Goodman et al. relates to plant cells and plants containing a gene encoding a mutant glutamine synthetase (GS) resistant to inhibition by herbicides that were known to inhibit GS, e.g. phosphinothricin and methionine sulfoximine. U.S. Pat. No. 5,013,659 to Bedbrook et al. is directed to plants expressing a mutant acetolactate synthase that renders the plants resistant to inhibition by sulfonylurea herbicides. U.S. Pat. No. 5,162,602 to Somers et al. discloses plants tolerant to inhibition by cyclohexanedione and aryloxyphenoxypropanoic acid herbicides. The tolerance is conferred by an altered acetyl coenzyme A carboxylase (ACCase).

Notwithstanding the above-described advancements, there remains a persistent and ongoing problem with unwanted or detrimental vegetation growth (e.g. weeds). Furthermore, as the population continues to grow, there will be increasing food shortages. Therefore, there exists a long felt, yet unfulfilled need, to find new, effective, and economic herbicides.

### SUMMARY OF THE INVENTION

It is an object of the invention to provide an effective and beneficial method to identify novel herbicides. A feature of the invention is the identification of a gene in A. thaliana, herein referred to as the 1917 gene, which shows sequence similarity to arginyl tRNA synthetase (Girjes et al. (1995) Gene, 164: 347-350; GenBank accession # Z98760 for this Arabidopsis gene). A feature of the invention is the identification of a gene in A. thaliana, herein referred to as the 2092 gene, which shows sequence similarity to alanyl tRNA synthetase (Mireau et al. (1996) The Plant Cell 8: 1027-1039). A feature of the invention is the identification of a gene in A. thaliana, herein referred to as the 7724 gene, which shows sequence similarity to 2′ tRNA phosphotransferase (Culver et al. (1997) J Biol Chemistry, 272:13203-13210; Spinelli et al. (1999) J. Biol. Chemistry, 274:2637-2644; Spinelli et al. (1997) RNA, 3:1388-1400). Another feature of the invention is the discovery that the 1917, 2092, and 7724 genes are essential for normal growth and development. An advantage of the present invention is that the newly discovered essential genes provide the basis for identity of a novel herbicidal mode of action which enables one skilled in the art to easily and rapidly discover novel inhibitors of gene function useful as herbicides.

One object of the present invention is to provide essential genes in plants for assay development for discovery of inhibitory compounds with herbicidal activity. Genetic results show that when any one of the 1917, 2092, or 7724 genes is mutated in Arabidopsis thaliana, the resulting phenotype is lethal in the homozygous state. This suggests a critical role for the gene products encoded by the 1917, 2092, and 7724 genes.

Using T-DNA insertion mutagenesis, the inventors of the present invention have demonstrated that the activity of each of the 1917, 2092, or 7724 gene products is essential for A. thaliana growth. This implies that chemicals, which inhibit the function of the 1917-, 2092-, or 7724-encoded proteins in plants, are likely to have detrimental effects on plants and are potentially good herbicide candidates. The present invention therefore provides methods of using a purified protein encoded by any of the 1917, 2092, or 7724 gene sequences described below to identify inhibitors thereof, which can then be used as herbicides to suppress the growth of undesirable vegetation, e.g. in fields where crops are grown, particularly agronomically important crops such as maize and other cereal crops such as wheat, oats, rye, sorghum, rice, barley, millet, turf and forage grasses, and the like, as well as cotton, sugar cane, sugar beet, oilseed rape, and soybeans.

The present invention discloses novel nucleotide sequences derived from A. thaliana, designated the 1917, 2092, or 7724 genes. The nucleotide sequences of the coding regions for the cDNA clones are set forth in SEQ ID NO:1, SEQ ID NO:3, and SEQ ID NO:5, respectively, and the corresponding amino acid sequences of the 1917-, 2092-, and 7724-encoded proteins are set forth in SEQ ID NO:2, SEQ ID NO:4, and SEQ ID NO:6, respectively. The present invention also includes nucleotide sequences substantially similar to those set forth in SEQ ID NO:1, SEQ ID NO:3, and SEQ ID NO:5, respectively. The present invention also encompasses plant proteins whose amino acid sequence are substantially similar to the amino acid sequences set forth in SEQ ID NO:2, SEQ ID NO:4, and SEQ ID NO:6, respectively. The present invention also includes methods of using the 1917, 2092, or 7724 gene products as herbicide targets, based on the essentiality of these genes for normal growth and development. Furthermore, the invention can be used in a screening assay to identify inhibitors of 1917, 2092, or 7724 gene function that are potential herbicides.

In a preferred embodiment, the present invention relates to a method for identifying chemicals having the ability to inhibit 1917, 2092, or 7724 activity in plants preferably comprising the steps of: a) obtaining transgenic plants, plant tissue, plant seeds or plant cells, preferably stably transformed, comprising a non-native nucleotide sequence encoding an enzyme having 1917, 2092, or 7724 activity and capable of overexpressing an enzymatically active 1917, 2092, or 7724 gene product (either full length or truncated but still active); b) applying a chemical to the transgenic plants, plant cells, tissues or parts and to the isogenic non-transformed plants, plant cells, tissues or parts; c) determining the growth or viability of the transgenic and non-transformed plants, plant cells, tissues after application of the chemical; d) comparing the growth or viability of the transgenic and non-transformed plants, plant cells, tissues after application of the chemical; and e) selecting chemicals that suppress the viability or growth of the non-transgenic plants, plant cells, tissues or parts, without significantly suppressing the growth of the viability or growth of the isogenic transgenic plants, plant cells, tissues or parts. In a preferred embodiment, the enzyme having 1917, 2092, or 7724 activity is encoded by a nucleotide sequence derived from a plant, preferably Arabidopsis thaliana, desirably identical or substantially similar to the nucleotide sequence set forth in SEQ ID NO:1, SEQ ID NO:3, and SEQ ID NO:5, respectively. In another embodiment, the enzyme having 1917, 2092, or 7724 activity is encoded by a nucleotide sequence capable of encoding the amino acid sequence of SEQ ID NO:2, SEQ ID NO:4, and SEQ ID NO:6, respectively. In yet another embodiment, the enzyme having 1917, 2092, or 7724 activity has an amino acid sequence identical or substantially similar to the amino acid sequence set forth in SEQ ID NO:2, SEQ ID NO:4, and SEQ ID NO:6, respectively.

The present invention further embodies plants, plant tissues, plant seeds, and plant cells that have modified 1917, 2092, or 7724 activity and that are therefore tolerant to inhibition by a herbicide at levels normally inhibitory to naturally occurring 1917, 2092, or 7724-encoded activity. Herbicide tolerant plants encompassed by the invention include those that would otherwise be potential targets for 1917, 2092, or 7724-inhibiting herbicides, particularly the agronomically important crops mentioned above. According to this embodiment, plants, plant tissue, plant seeds, or plant cells are transformed, preferably stably transformed, with a recombinant DNA molecule comprising a suitable promoter functional in plants operatively linked to a nucleotide sequence that encodes a modified 1917, 2092, or 7724 gene that is tolerant to inhibition by a herbicide at a concentration that would normally inhibit the activity of wild-type, unmodified 1917, 2092, or 7724 gene product. Modified 1917, 2092, or 7724 activity may also be conferred upon a plant by increasing expression of wild-type herbicide-sensitive 1917, 2092, or 7724 protein by providing multiple copies of wild-type 1917, 2092, or 7724 genes to the plant or by overexpression of wild-type 1917, 2092, or 7724 genes under control of a stronger-than-wild-type promoter. The transgenic plants, plant tissue, plant seeds, or plant cells thus created are then selected using conventional techniques, whereby herbicide tolerant lines are isolated, characterized, and developed. Alternately, random or site-specific mutagenesis may be used to generate herbicide tolerant lines.

Therefore, the present invention provides a plant, plant cell, plant seed, or plant tissue transformed with a DNA molecule comprising a nucleotide sequence isolated from a plant that encodes an enzyme having 1917, 2092, or 7724 activity, wherein the DNA expresses the 1917, 2092, or 7724 activity and wherein the DNA molecule confers upon the plant, plant cell, plant seed, or plant tissue tolerance to a herbicide in amounts that normally inhibits naturally occurring 1917, 2092, or 7724 activity. According to one example of this embodiment, the enzyme having 1917, 2092, or 7724 activity is encoded by a nucleotide sequence identical or substantially similar to the nucleotide sequence set forth in SEQ ID NO:1, SEQ ID NO:3, and SEQ ID NO:5, respectively, or has an amino acid sequence identical or substantially similar to the amino acid sequence set forth in SEQ ID NO:2, SEQ ID NO:4, and SEQ ID NO:6, respectively.

The invention also provides a method for suppressing the growth of a plant comprising the step of applying to the plant a chemical that inhibits the naturally occurring 1917, 2092, or 7724 activity in the plant. In a related aspect, the present invention is directed to a method for selectively suppressing the growth of undesired vegetation in a field containing a crop of planted crop seeds or plants, comprising the steps of: (a) optionally planting herbicide tolerant crops or crop seeds, which are plants or plant seeds that are tolerant to a herbicide that inhibits the naturally occurring 1917, 2092, or 7724 activity; and (b) applying to the herbicide tolerant crops or crop seeds and the undesired vegetation in the field a herbicide in amounts that inhibit naturally occurring 1917, 2092, or 7724 activity, wherein the herbicide suppresses the growth of the weeds without significantly suppressing the growth of the crops.

The invention thus provides an isolated DNA molecule comprising a nucleotide sequence substantially similar to SEQ ID NO:1, SEQ ID NO:3, or SEQ ID NO:5, respectively. In a preferred embodiment, the nucleotide sequence encodes an amino acid sequence substantially similar to SEQ ID NO:2, SEQ ID NO:4, or SEQ ID NO:6, respectively. In another preferred embodiment, the nucleotide sequence is SEQ ID NO:1, SEQ ID NO:3, or SEQ ID NO:5, respectively. In yet another preferred embodiment, the nucleotide sequence encodes the amino acid sequence of SEQ ID NO:2, SEQ ID NO:4, or SEQ ID NO:6, respectively. Preferably, the nucleotide sequence is a plant nucleotide sequence, which preferably encodes a polypeptide having 1917, 2092, or 7724 activity, respectively.

The invention further provides a polypeptide comprising an amino acid sequence encoded by a nucleotide sequence substantially similar to SEQ ID NO:1, SEQ ID NO:3, or SEQ ID NO:5, respectively. Preferably, the amino acid sequence is encoded by SEQ ID NO:1, SEQ ID NO:3, or SEQ ID NO:5, respectively. Preferably, the polypeptide comprises an amino acid sequence substantially similar to SEQ ID NO:2, SEQ ID NO:4, or SEQ ID NO:6, respectively. Preferably the amino acid sequence is SEQ ID NO:2, SEQ ID NO:4, or SEQ ID NO:6respectively. The amino acid sequence preferably has 1917, 2092, or 7724 activity, respectively. In another preferred embodiment, the amino acid sequence comprises at least 20 consecutive amino acid residues of the amino acid sequence encoded by SEQ ID NO:1, SEQ ID NO:3, or SEQ ID NO:5, respectively. Or, alternatively, the amino acid sequence comprises at least 20 consecutive amino acid residues of the amino acid sequence of SEQ ID NO:2, SEQ ID NO:4, or SEQ I) NO:6, respectively.

The invention further provides an expression cassette comprising a promoter operatively linked to a DNA molecule according to the present invention, a recombinant vector comprising an expression cassette according to the present invention, wherein said vector is preferably capable of being stably transformed into a host cell, a host cell comprising a DNA molecule according to the present invention, wherein said DNA molecule is preferably expressible in the cell. The host cell is preferably selected from the group consisting of an insect cell, a yeast cell, a prokaryotic cell and a plant cell. The invention further provides a plant or seed comprising a plant cell of the present invention, wherein the plant or seed is preferably tolerant to an inhibitor of 1917, 2092, or 7724 activity, respectively.

The invention further provides a process for making nucleotide sequences encoding gene products having altered 1917, 2092, or 7724 activity, respectively, comprising: a) shuffling an unmodified nucleotide sequence of the present invention, b) expressing the resulting shuffled nucleotide sequences, and c) selecting for altered 1917, 2092, or 7724 activity, respectively, as compared to the 1917, 2092, or 7724 activity, respectively, of the gene product of said unmodified nucleotide sequence.

In a preferred embodiment, the unmodified nucleotide sequence is identical or substantially similar to SEQ ID NO:1, SEQ ID NO:3, or SEQ ID NO:5, respectively, or a homolog thereof. The present invention further provides a DNA molecule comprising a shuffled nucleotide sequence obtainable by the process described above, a DNA molecule comprising a shuffled nucleotide sequence produced by the process described above. Preferably, a shuffled nucleotide sequence obtained by the process described above has enhanced tolerance to an inhibitor of 1917, 2092, or 7724 activity, respectively. The invention further provides an expression cassette comprising a promoter operatively linked to a DNA molecule comprising a shuffled nucleotide sequence a recombinant vector comprising such an expression cassette, wherein said vector is preferably capable of being stably transformed into a host cell, a host cell comprising such an expression cassette, wherein said nucleotide sequence is preferably expressible in said cell. A preferred host cell is selected from the group consisting of an insect cell, a yeast cell, a prokaryotic cell and a plant cell. The invention further provides a plant or seed comprising such plant cell, wherein the plant is preferably tolerant to an inhibitor of 1917, 2092, or 7724 activity, respectively.

The invention further provides a method for selecting compounds that interact with the protein encoded by SEQ ID NO:1, SEQ ID NO:3, or SEQ ID NO:5, respectively, comprising: a) expressing a DNA molecule comprising SEQ ID NO:1, SEQ ID NO:3, or SEQ ID NO:5, respectively, or a sequence substantially similar to SEQ ID NO: 1, SEQ ID NO:3, or SEQ ID NO:5, respectively, or a homolog thereof, to generate the corresponding protein, b) testing a compound suspected of having the ability to interact with the protein expressed in step (a), and (c) selecting compounds that interact with the protein in step (b).

The invention further provides a process of identifying an inhibitor of 1917, 2092, or 7724 activity, respectively, comprising: a) introducing a DNA molecule comprising a nucleotide sequence of SEQ ID NO:1, SEQ ID NO:3, or SEQ ID NO:5, respectively, and having 1917, 2092, or 7724 activity, respectively, or nucleotide sequences substantially similar thereto, or a homolog thereof, into a plant cell, such that said sequence is functionally expressible at levels that are higher than wild-type expression levels, b) combining said plant cell with a compound to be tested for the ability to inhibit the 1917, 2092, or 7724 activity, respectively, under conditions conducive to such inhibition, c) measuring plant cell growth under the conditions of step (b), d) comparing the growth of said plant cell with the growth of a plant cell having unaltered 1917, 2092, or 7724 activity, respectively, under identical conditions, and e) selecting said compound that inhibits plant cell growth in step (d).

The invention further comprises a compound having herbicidal activity identifiable according to the process described immediately above.

The invention further comprises:

A process of identifying compounds having herbicidal activity comprising: a) combining a protein of the present invention and a compound to be tested for the ability to interact with said protein, under conditions conducive to interaction, b) selecting a compound identified in step (a) that is capable of interacting with said protein, c) applying identified compound in step (b) to a plant to test for herbicidal activity, and d) selecting compounds having herbicidal activity.

The invention further comprises a compound having herbicidal activity identifiable according to the process described immediately above.

The invention further comprises:

A method for suppressing the growth of a plant comprising, applying to said plant a compound that inhibits the activity of a polypeptide of the present invention in an amount sufficient to suppress the growth of said plant.

The invention further comprises:

A method for recombinantly expressing a protein having 1917, 2092, or 7724 activity comprising introducing a nucleotide sequence encoding a protein having one of the above activities into a host cell and expressing the nucleotide sequence in the host cell. A preferred host cell is selected from the group consisting of an insect cell, a yeast cell, a prokaryotic cell and a plant cell. A preferred prokaryotic cell is a bacterial cell, e.g. E. coli.

Other objects and advantages of the present invention will become apparent to those skilled in the art from a study of the following description of the invention and non-limiting examples.

### Definitions

For clarity, certain terms used in the specification are defined and presented as follows:

Cofactor: natural reactant, such as an organic molecule or a metal ion, required in an enzyme-catalyzed reaction. A co-factor is e.g. NAD(P), riboflavin (including FAD and FMN), folate, molybdopterin, thiamin, biotin, lipoic acid, pantothenic acid and coenzyme A, S-adenosylmethionine, pyridoxal phosphate, ubiquinone, menaquinone. Optionally, a co-factor can be regenerated and reused.

DNA shuffling: DNA shuffling is a method to rapidly, easily and efficiently introduce mutations or rearrangements, preferably randomly, in a DNA molecule or to generate exchanges of DNA sequences between two or more DNA molecules, preferably randomly. The DNA molecule resulting from DNA shuffling is a shuffled DNA molecule that is a non-naturally occurring DNA molecule derived from at least one template DNA molecule. The shuffled DNA encodes an enzyme modified with respect to the enzyme encoded by the template DNA, and preferably has an altered biological activity with respect to the enzyme encoded by the template DNA.

Enzyme activity: means herein the ability of an enzyme to catalyze the conversion of a substrate into a product. A substrate for the enzyme comprises the natural substrate of the enzyme but also comprises analogues of the natural substrate which can also be converted by the enzyme into a product or into an analogue of a product. The activity of the enzyme is measured for example by determining the amount of product in the reaction after a certain period of time, or by determining the amount of substrate remaining in the reaction mixture after a certain period of time. The activity of the enzyme is also measured by determining the amount of an unused co-factor of the reaction remaining in the reaction mixture after a certain period of time or by determining the amount of used co-factor in the reaction mixture after a certain period of time. The activity of the enzyme is also measured by determining the amount of a donor of free energy or energy-rich molecule (e.g. ATP, phosphoenolpyruvate, acetyl phosphate or phosphocreatine) remaining in the reaction mixture after a certain period of time or by determining the amount of a used donor of free energy or energy-rich molecule (e.g. ADP, pyruvate, acetate or creatine) in the reaction mixture after a certain period of time.

Herbicide: a chemical substance used to kill or suppress the growth of plants, plant cells, plant seeds, or plant tissues.

Heterologous DNA Sequence: a DNA sequence not naturally associated with a host cell into which it is introduced, including non-naturally occurring multiple copies of a naturally occurring DNA sequence; and genetic constructs wherein an otherwise homologous DNA sequence is operatively linked to a non-native sequence.

Homologous DNA Sequence: a DNA sequence naturally associated with a host cell into which it is introduced.

Inhibitor: a chemical substance that causes abnormal growth, e.g., by inactivating the enzymatic activity of a protein such as a biosynthetic enzyme, receptor, signal transduction protein, structural gene product, or transport protein that is essential to the growth or survival of the plant. In the context of the instant invention, an inhibitor is a chemical substance that alters the enzymatic activity encoded by a nucleotide sequence of the present invention. More generally, an inhibitor causes abnormal growth of a host cell by interacting with the gene product encoded by the nucleotide sequence of the present invention.

Isogenic: plants which are genetically identical, except that they may differ by the presence or absence of a heterologous DNA sequence.

Isolated: in the context of the present invention, an isolated DNA molecule or an isolated enzyme is a DNA molecule or enzyme that, by the hand of man, exists apart from its native environment and is therefore not a product of nature. An isolated DNA molecule or enzyme may exist in a purified form or may exist in a non-native environment such as, for example, in a transgenic host cell.

Mature protein: protein which is normally targeted to a cellular organelle, such as a chloroplast, and from which the transit peptide has been removed.

Minimal Promoter: promoter elements, particularly a TATA element, that are inactive or that have greatly reduced promoter activity in the absence of upstream activation. In the presence of a suitable transcription factor, the minimal promoter functions to permit transcription.

Modified Enzyme Activity: enzyme activity different from that which naturally occurs in a plant (i.e. enzyme activity that occurs naturally in the absence of direct or indirect manipulation of such activity by man), which is tolerant to inhibitors that inhibit the naturally occurring enzyme activity.

Pre-protein: protein which is normally targeted to a cellular organelle, such as a chloroplast, and still comprising its transit peptide.

Significant Increase: an increase in enzymatic activity that is larger than the margin of error inherent in the measurement technique, preferably an increase by about 2-fold or greater of the activity of the wild-type enzyme in the presence of the inhibitor, more preferably an increase by about 5-fold or greater, and most preferably an increase by about 10-fold or greater.

Significantly less: means that the amount of a product of an enzymatic reaction is reduced by more than the margin of error inherent in the measurement technique, preferably a decrease by about 2-fold or greater of the activity of the wild-type enzyme in the absence of the inhibitor, more preferably an decrease by about 5-fold or greater, and most preferably an decrease by about 10-fold or greater.

Substantially similar: with respect to a gene of the present invention, in its broadest sense, the term “substantially similar”, when used herein with respect to a nucleotide sequence, means a nucleotide sequence corresponding to a reference nucleotide sequence, wherein the corresponding sequence encodes a polypeptide having substantially the same structure and function as the polypeptide encoded by the reference nucleotide sequence, e.g. where only changes in amino acids not affecting the polypeptide function occur. Desirably the substantially similar nucleotide sequence encodes the polypeptide encoded by the reference nucleotide sequence. The term “substantially similar” is specifically intended to include nucleotide sequences wherein the sequence has been modified to optimize expression in particular cells. A nucleotide sequence “substantially similar” to a reference nucleotide sequence has a complement that hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C. As used herein the term “1917 gene” refers to a DNA molecule comprising SEQ ID NO: 1 or comprising a nucleotide sequence substantially similar to SEQ ID NO: 1. As used herein the term “2092 gene” refers to a DNA molecule comprising SEQ ID NO:3 or comprising a nucleotide sequence substantially similar to SEQ ID NO:3. As used herein the term “7724 gene” refers to a DNA molecule comprising SEQ ID NO:5 or comprising a nucleotide sequence substantially similar to SEQ ID NO:5.

With respect to a protein of the present invention, the term “substantially similar”, when used herein with respect to a protein, means a protein corresponding to a reference protein, wherein the protein has substantially the same structure and function as the reference protein, e.g. where only changes in amino acids sequence not affecting the polypeptide function occur.

One skilled in the art is also familiar with analysis tools, such as GAP analysis, to determine the percentage of identity between the “substantially similar” and the reference nucleotide sequence, or protein or amino acid sequence. In the present invention, “substantially similar” is therefore also determined using default GAP analysis parameters with the University of Wisconsin GCG, SEQWEB application of GAP, based on the algorithm of Needleman and Wunsch (Needleman and Wunsch (1970) J Mol. Biol. 48: 443-453).

Thus, in the context of the “1917 gene” and using GAP analysis as described above, “substantially similar” refers to nucleotide sequences that encode a protein having at least 48% identity, more preferably at least 50% identity, still more preferably at least 65% identity, still more preferably at least 75% identity, still more preferably at least 85% identity, still more preferably at least 95% identity, yet still more preferably at least 99% identity to SEQ ID NO:2. Further, using GAP analysis as described above, “homologs of the 1917 gene” include nucleotide sequences that encode an amino acid sequence that has at least 30% identity to SEQ ID NO:2, more preferably at least 40% identity, still more preferably at least 45% identity, still more preferably at least 55% identity, yet still more preferably at least 65% identity, still more preferably at least 75% identity, yet still more preferably at least 85% identity to SEQ ID NO:2, wherein the amino acid sequence encoded by the homolog has the biological activity of the 1917 protein.

When using GAP analysis as described above with respect to a protein or an amino acid sequence and in the context of the “1917 gene”, the percentage of identity between the “substantially similar” protein or amino acid sequence and the reference protein or amino acid sequence (in this case SEQ ID NO:2) is at least 48%, more preferably at least 50%, still more preferably at least 65%, still more preferably at least 75%, still more preferably at least 85%, still more preferably at least 95%, yet still more preferably at least 99%.

“Homologs of the 1917 protein” include amino acid sequences that are at least 30% identical to SEQ ID NO:2, more preferably at least 40% identical, still more preferably at least 45% identical, still more preferably at least 55% identical, yet still more preferably at least 65% identical, still more preferably at least 75% identical, yet still more preferably at least 85% identical to SEQ ID NO:2, wherein homologs of the 1917 protein have the biological activity of the 1917 protein.

Thus, in the context of the “2092 gene” and using GAP analysis as described above, “substantially similar” refers to nucleotide sequences that encode a protein having at least 58% identity, more preferably at least 65% identity, still more preferably at least 75% identity, still more preferably at least 85% identity, still more preferably at least 95% identity, yet still more preferably at least 99% identity to SEQ ID NO:4. Further, using GAP analysis as described above, “homologs of the 2092 gene” include nucleotide sequences that encode an amino acid sequence that has at least 34% identity to SEQ ID NO:4, more preferably at least 40% identity, still more preferably at least 50% identity, still more preferably at least 60% identity, yet still more preferably at least 65% identity, still more preferably at least 75% identity, yet still more preferably at least 85% identity to SEQ ID NO:4, wherein the amino acid sequence encoded by the homolog has the biological activity of the 2092 protein.

When using GAP analysis as described above with respect to a protein or an amino acid sequence and in the context of the “2092 gene”, the percentage of identity between the “substantially similar” protein or amino acid sequence and the reference protein or amino acid sequence (in this case SEQ ID NO:4) is at least 58%, more preferably at least 65%, still more preferably at least 75%, still more preferably at least 85%, still more preferably at least 95%, yet still more preferably at least 99%.

“Homologs of the 2092 protein” include amino acid sequences that are at least 34% identical to SEQ ID NO:4, more preferably at least 50% identical, still more preferably at least 55% identical, still more preferably at least 60% identical, yet still more preferably at least 65% identical, still more preferably at least 75% identical, yet still more preferably at least 85% identical to SEQ ID NO:4, wherein homologs of the 2092 protein have the biological activity of the 2092 protein.

Thus, in the context of the “7724 gene” and using GAP analysis as described above, “substantially similar” refers to nucleotide sequences that encode a protein having at least 36% identity, more preferably at least 50% identity, more preferably at least 70% identity, more preferably at least 90% identity, still more preferably at least 99% identity to SEQ ID NO:6. Further, using GAP analysis as described above, “homologs of the 7724 gene” include nucleotide sequences that encode an amino acid sequence that has at least 30% identity to SEQ ID NO:6, more preferably at least 40% identity, still more preferably at least 50% identity, still more preferably at least 60% identity, yet still more preferably at least 70% identity, still more preferably at least 85% identity, yet still more preferably at least 90% identity to SEQ ID NO:6, wherein the amino acid sequence encoded by the homolog has the biological activity of the 7724 protein.

When using GAP analysis as described above with respect to a protein or an amino acid sequence and in the context of the “7724 gene”, the percentage of identity between the “substantially similar” protein or amino acid sequence and the reference protein or amino acid sequence (in this case SEQ ID NO:6) is at least 36%, more preferably at least 50% identity, more preferably at least 70% identity, more preferably at least 90% identity, still more preferably at least 99%.

“Homologs of the 7724 protein” include amino acid sequences that are at least 30% identical to SEQ ID NO:6, more preferably at least 40% identical, still more preferably at least 50% identical, still more preferably at least 60% identical, yet still more preferably at least 70% identical, still more preferably at least 85% identical, yet still more preferably at least 95% identical to SEQ ID NO:6, wherein homologs of the 7724 protein have the biological activity of the 7724 protein.

Substrate: a substrate is the molecule that an enzyme naturally recognizes and converts to a product in the biochemical pathway in which the enzyme naturally carries out its function, or is a modified version of the molecule, which is also recognized by the enzyme and is converted by the enzyme to a product in an enzymatic reaction similar to the naturally-occurring reaction.

Tolerance: the ability to continue essentially normal growth or function when exposed to an inhibitor or herbicide in an amount sufficient to suppress the normal growth or function of native, unmodified plants.

Transformation: a process for introducing heterologous DNA into a cell, tissue, or plant. Transformed cells, tissues, or plants are understood to encompass not only the end product of a transformation process, but also transgenic progeny thereof.

Transgenic: stably transformed with a recombinant DNA molecule that preferably comprises a suitable promoter operatively linked to a DNA sequence of interest.

### BRIEF DESCRIPTION OF THE SEQUENCES IN THE SEQUENCE LISTING

SEQ ID NO:1 cDNA coding sequence for isoform II of the Arabidopsis thaliana 1917 gene

SEQ ID NO:2 amino acid sequence encoded by isoform II of the Arabidopsis thaliana 1917 DNA sequence shown in SEQ ID NO:1

SEQ ID NO:3 cDNA coding sequence for the Arabidopsis thaliana 2092 gene

SEQ ID NO:4 amino acid sequence encoded by the Arabidopsis thaliana 2092 cDNA sequence shown in SEQ ID NO:3

SEQ ID NO:5 cDNA coding sequence for the Arabidopsis thaliana 7724 gene

SEQ ID NO:6 amino acid sequence encoded by the Arabidopsis thaliana 7724 DNA sequence shown in SEQ ID NO:5

SEQ ID NO:7 complete cDNA coding sequence, including 5′ UTR, coding region, and 3′ UTR sequences, for the Arabidopsis thaliana 2092 gene

SEQ ID NO:8 amino acid sequence encoded by the Arabidopsis thaliana 2092 DNA sequence shown in SEQ ID NO:7

SEQ ID NO:9 oligonucleotide CA50

SEQ ID NO:10 oligonucleotide CA51

SEQ ID NO:11 oligonucleotide CA52

SEQ ID NO:12 oligonucleotide CA53

SEQ ID NO:13 oligonucleotide CA54

SEQ ID NO:14 oligonucleotide CA55

SEQ ID NO:15 oligonucleotide CA66

SEQ ID NO:16 oligonucleotide CA67

SEQ ID NO:17 oligonucleotide CA68

SEQ ID NO:18 oligonucleotide JM33

SEQ ID NO:19 oligonucleotide JM34

SEQ ID NO:20 oligonucleotide JM35

SEQ ID NO:21 complete cDNA coding sequence, including 5′ UTR, coding region, and 3′ UTR sequences, for the Arabidopsis thaliana 7724 gene

SEQ ID NO:22 amino acid sequence encoded by the Arabidopsis thaliana 7724 DNA sequence shown in SEQ ID NO:21

SEQ ID NO:23 genomic sequence of the Arabidopsis thaliana 7724 gene

SEQ ID NO:24 cDNA coding sequence for isoform I of the Arabidopsis thaliana 1917 gene

SEQ ID NO:25 amino acid sequence encoded by isoform I of the Arabidopsis thaliana 1917 DNA sequence shown in SEQ ID NO:24

SEQ ID NO:26 oligonucleotide slp346

SEQ ID NO:27 oligonucleotide JM99

SEQ ID NO:28 oligonucleotide JM100

### DETAILED DESCRIPTION OF THE INVENTION

I.a. Essentiality of the 1917, 2092, and 7724 Genes in Arabidopsis thaliana Demonstrated by T-DNA Insertion Mutagenesis

As shown in the examples below, the identification of a novel gene structure, as well as the essentiality of the 1917, 2092, and 7724 genes for normal plant growth and development, have been demonstrated for the first time in Arabidopsis using T-DNA insertion mutagenesis. Having established the essentiality of 1917, 2092, and 7724 function in plants and having identified the genes encoding these essential activities, the inventors thereby provide an important and sought after tool for new herbicide development.

Essential genes are identified through the isolation of lethal mutants blocked in early development. Examples of lethal mutants include those blocked in the formation of the male or female gametes or embryo. Gametophytic mutants are found by examining T1 insertion lines for the presence of 50% aborted pollen grains or ovules. Embryo defective mutants produce 25% defective seeds following self-pollination of T1 plants (see Errampalli et al. 1991, Plant Cell 3:149-157; Castle et al. 1993, Mol Gen Genet 241:504-514).

When a line is identified as segregating for an embryo lethal mutation, it is determined if the resistance marker in the T-DNA co-segregates with the lethality (Errampalli et al. (1991) The Plant Cell, 3:149-157). Cosegregation analysis is done by placing the seeds on media containing the selective agent and scoring the seedlings for resistance or sensitivity to the agent. Examples of selective agents used are hygromycin or phosphinothricin. About (these are the actual numbers) 17 (1917), 35 (2092), and 37 (7724) resistant seedlings are transplanted to soil and their progeny are examined for the segregation of the embryo-lethal phenotype. In the case in which the T-DNA insertion disrupts an essential gene, there is cosegregation of the resistance phenotype and the embryo-lethal phenotype in every plant. Therefore, in such a case, all resistant plants segregate for the lethal phenotype in the next generation; this result indicates that each of the resistant plants is heterozygous for the mutation and hemizygous for the T-DNA insert causing the mutation. For those lines showing cosegregation of the T-DNA resistance marker and the lethal phenotype, PCR-based approaches, such as TAIL PCR (Liu and Whittier (1995), Genomics, 25: 674-681) vectorette PCR (Riley et al. (1990) Nucleic Acids Research, 18: 2887-2890), or a strategy such as the Genome Walker system (CLONTECH Laboratories, Inc, Palo Alto, Calif.), may be used to directly amplify plant DNA/T-DNA border fragments. Each of these techniques takes advantage of the fact that the DNA sequence of the insertion element is known, and can routinely be used to recover small (less than 5 kb) fragments adjacent to the known sequence. Alternatively, plasmid rescue may be used to isolate the plant DNA/T-DNA border fragments. Southern blot analysis may be performed as an initial step in the characterization of the molecular nature of each insertion. Southern blots are done with genomic DNA isolated from heterozygotes and using probes capable of hybridizing with the T-DNA vector DNA.

Using the results of the Southern analysis, appropriate restriction enzymes are chosen to perform plasmid rescue in order to molecularly clone Arabidopsis thaliana genomic DNA flanking one or both sides of the T-DNA insertion. Plasmids obtained in this manner are analyzed by restriction enzyme digestion to sort the plasmids into classes based on their digestion pattern. For each class of plasmid clone, the DNA sequence is determined.

The resulting sequences, obtained by any of the above outlined approaches, are analyzed for the presence of non-T-DNA vector sequences. When such sequences are found, they are used to search DNA and protein databases using the BLAST and BLAST2 programs (Altschul et al. (1990) J Mol. Biol. 215: 403-410; Altschul et al (1997) Nucleic Acid Res. 25:3389-3402). Additional genomic and cDNA sequences for each gene are identified by standard molecular biology procedures.

One method of confirming that the disrupted gene is the cause of the mutant phenotype is to transform a wild-type form of the gene into the mutant plant. Another method is identification of a second mutant allele showing a lethal phenotype. Alternatively, the mutant is phenocopied by specifically reducing expression of the disrupted gene in transgenic plants expressing an antisense version of the gene behind a synthetic promoter (Guyer et al. (1998) Genetics, 149: 633-639).

II. Sequence of the Arabidopsis 1917, 2092, and 7724 Gene

The Arabidopsis 1917 gene is identified by isolating DNA flanking the T-DNA border from the tagged embryo-lethal line # 1917. Arabidopsis DNA flanking the T-DNA border is identical to regions of two sequenced EST clones from Arabidopsis (Genbank accession numbers H77096 and R30603). The inventors are the first to demonstrate that the 1917 gene product is essential for normal growth and development in plants, as well as defining the function of the 1917 gene product through protein homology. The present invention discloses the cDNA nucleotide sequence of the Arabidopsis 1917 gene as well as the amino acid sequence of the Arabidopsis 1917 protein. The nucleotide sequence corresponding to the cDNA coding region is set forth in SEQ ID NO:1, and the amino acid sequence encoding the protein is set forth in SEQ ID NO:2. The nucleotide sequence corresponding to the complete cDNA, which includes 5′ UTR and coding and 3′ UTR sequences, is set forth in SEQ ID NO:24. The present invention also encompasses an isolated amino acid sequence derived from a plant, wherein said amino acid sequence is identical or substantially similar to the amino acid sequence encoded by the nucleotide sequence set forth in SEQ ID NO: 1, wherein said amino acid sequence has 1917 activity. Using GAP programs with the default settings, the sequence of the 1917 gene shows similarity to arginyl tRNA synthetase. Notable species similarities include: chinese hamster (Genbank peptide accession # P37880); human (Genbank peptide accession #NP_{—}002878.1); Synechocystis (Genbank peptide accession # Q55486); C. elegans (Genbank peptide accession # Q19825); Chlamydia sp. (Genbank peptide accession # AE001641); Streptomyces sp. (Genbank peptide accession # AL079345); Haemophilus (Genbank peptide accession # P43832); E. coli (Genbank peptide accession # P11875); S. cerevisiae (Genbank peptide accession # NP_{—}010628.1); and S. pombe (Genbank peptide accession # AL031853).

The Arabidopsis 2092 gene is identified by isolating DNA flanking the T-DNA border from the tagged embryo-lethal line # 2092. Arabidopsis DNA flanking the T-DNA border is identical to a sequenced P1 clone MRN17 (GenBank accession # AB005243). The inventors are the first to demonstrate that the 2092 gene product is essential for normal growth and development in plants, as well as defining the function of the 2092 gene product through protein homology. The present invention discloses the cDNA nucleotide sequence of the Arabidopsis 2092 gene as well as the amino acid sequence of the Arabidopsis 2092 protein. The nucleotide sequence corresponding to the cDNA coding region is set forth in SEQ ID NO:3, and the amino acid sequence encoding the protein is set forth in SEQ ID NO:4. The present invention also encompasses an isolated amino acid sequence derived from a plant, wherein said amino acid sequence is identical or substantially similar to the amino acid sequence encoded by the nucleotide sequence set forth in SEQ ID NO: 4, wherein said amino acid sequence has 2092 activity. Using GAP programs with the default settings, the sequence of the 2092 gene shows similarity to alanyl tRNA synthetase genes. Notable species similarities include: Synechocystis (Genbank peptide accession # G2500959); E. coli (Genbank peptide accession # AE000353); yeast (Genbank peptide accession # NP_{—}014980); Drosophila (Genbank peptide accession # AF188718);, and human (Genbank peptide accession # AB033096).

The Arabidopsis 7724 gene is identified by isolating DNA flanking the T-DNA border from the tagged embryo-lethal line #7724. Arabidopsis DNA flanking the T-DNA border is identical to a portion of sequence to the BAC clone F4L23 (Genbank accession # AC002387). Annotation suggests that a gene is present in the region disrupted by the T-DNA. BLAST-N searches using default settings, using the annotated gene region reveals public EST clones with sequence identity to the predicted gene, indicating that this region contains an expressed gene. The EST clones are 10409T7 and 10409XP (different ends of the same clone). The inventors are the first to demonstrate that the 7724 gene product is essential for normal growth and development in plants, as well as defining the function of the 7724 gene product through protein homology. The present invention discloses the cDNA nucleotide sequence of the Arabidopsis 7724 gene as well as the amino acid sequence of the Arabidopsis 7724 protein. The nucleotide sequence corresponding to the cDNA coding region is set forth in SEQ ID NO:5, and the amino acid sequence encoding the protein is set forth in SEQ ID NO:6. The present invention also encompasses an isolated amino acid sequence derived from a plant, wherein said amino acid sequence is identical or substantially similar to the amino acid sequence encoded by the nucleotide sequence set forth in SEQ ID NO: 5, wherein said amino acid sequence has 7724 activity. Using GAP programs with the default settings, the sequence of the 7724 gene shows similarity to 2′ tRNA phosphotransferase genes. Notable species similarities include: S. cerevisiae (Genbank peptide accession # NP_{—}014539); Streptomyces coelicolor (Genbank peptide accession # CAA22225); S. pombe (Genbank peptide accession # CAB16372); Pyrococcus horikoshii (Genbank peptide accession # BAA29229); and Archaeoglobus fulgidus (Genbank peptide accession number AAB90829).

III. Recombinant Production of 1917, 2092, and 7724 Activities and Uses Thereof

For recombinant production of 1917, 2092, or 7724 activity in a host organism, a nucleotide sequence encoding a protein having one of the above activities is inserted into an expression cassette designed for the chosen host and introduced into the host where it is recombinantly produced. For example, SEQ ID NO:1, or nucleotide sequences substantially similar to SEQ ID NO:1, or homologs of the 1917 coding sequence can be used for the recombinant production of a protein having 1917 activity. For example, SEQ ID NO:3, or nucleotide sequences substantially similar to SEQ ID NO:3, or homologs of the 2092 coding sequence can be used for the recombinant production of a protein having 2092 activity. For example, SEQ ID NO:5, or nucleotide sequences substantially similar to SEQ ID NO:5, or homologs of the 7724 coding sequence can be used for the recombinant production of a protein having 7724 activity. The choice of specific regulatory sequences such as promoter, signal sequence, 5′ and 3′ untranslated sequences, and enhancer appropriate for the chosen host is within the level of skill of the routineer in the art. The resultant molecule, containing the individual elements operably linked in proper reading frame, may be inserted into a vector capable of being transformed into the host cell. Suitable expression vectors and methods for recombinant production of proteins are well known for host organisms such as E. coli, yeast, and insect cells (see, e.g., Luckow and Summers, Bio/Technol. 6: 47 (1988), and baculovirus expression vectors, e.g., those derived from the genome of Autographica californica nuclear polyhedrosis virus (AcMNPV). A preferred baculovirus/insect system is pAcHLT (Pharmingen, San Diego, Calif.) used to transfect Spodoptera frugiperda Sf9 cells (ATCC) in the presence of linear Autographa californica baculovirus DNA (Pharmigen, San Diego, Calif.). The resulting virus is used to infect HighFive Tricoplusia ni cells (Invitrogen, La Jolla, Calif.).

In a preferred embodiment, the nucleotide sequence encoding a protein having 1917, 2092, or 7724 activity is derived from a eukaryote, such as a mammal, a fly or a yeast, but is preferably derived from a plant. In a further preferred embodiment, the nucleotide sequence is identical or substantially similar to the nucleotide sequence set forth in SEQ ID NO: 1, SEQ ID NO:3, or SEQ ID NO:5, respectively, or encodes a protein having 1917, 2092, or 7724 activity, respectively, whose amino acid sequence is identical or substantially similar to the amino acid sequence set forth in SEQ ID NO:2, SEQ ID NO:4, or SEQ ID NO:6, respectively. The nucleotide sequence set forth in SEQ ID NO:1 encodes the Arabidopsis 1917 protein, whose amino acid sequence is set forth in SEQ ID NO:2. The nucleotide sequence set forth in SEQ ID NO:3 encodes the Arabidopsis 2092 protein, whose amino acid sequence is set forth in SEQ ID NO:4. The nucleotide sequence set forth in SEQ ID NO:5 encodes the Arabidopsis 7724 protein, whose amino acid sequence is set forth in SEQ ID NO:6. In another preferred embodiment, the nucleotide sequences are derived from a prokaryote, preferably a bacteria, e.g. E. coli. Recombinantly produced protein having 1917, 2092, or 7724 activity is isolated and purified using a variety of standard techniques. The actual techniques that may be used will vary depending upon the host organism used, whether the protein is designed for secretion, and other such factors familiar to the skilled artisan (see, e.g. chapter 16 of Ausubel, F. et al., “Current Protocols in Molecular Biology”, pub. by John Wiley & Sons, Inc. (1994).

Assays Utilizing the 1917, 2092, or 7724 Protein

Recombinantly produced 1917, 2092, or 7724 proteins having 1917, 2092, or 7724 activities, respectively, are useful for a variety of purposes. For example, they can be used in in vitro assays to screen known herbicidal chemicals whose target has not been identified to determine if they inhibit 1917, 2092, or 7724. Such in vitro assays may also be used as more general screens to identify chemicals that inhibit such enzymatic activity and that are therefore novel herbicide candidates. Alternatively, recombinantly produced 1917, 2092, or 7724 proteins having 1917, 2092, or 7724 activity may be used to elucidate the complex structure of these molecules and to further characterize their association with known inhibitors in order to rationally design new inhibitory herbicides as well as herbicide tolerant forms of the enzymes.

In vitro Inhibitor Assays: Discovery of Small Molecule Ligand that Interacts with the Gene Product of SEQ ID NO:1, SEQ ID NO:3, or SEQ ID NO:5

Once a protein has been identified as a potential herbicide target, the next step is to develop an assay that allows screening large number of chemicals to determine which ones interact with the protein. Although it is straightforward to develop assays for proteins of known function, developing assays with proteins of unknown functions is more difficult.

This difficulty can be overcome by using technologies that can detect interactions between a protein and a compound without knowing the biological function of the protein. A short description of three methods is presented, including fluorescence correlation spectroscopy, surface-enhanced laser desorption/ionization, and biacore technologies.

Fluorescence Correlation Spectroscopy (FCS) theory was developed in 1972 but it is only in recent years that the technology to perform FCS became available (Madge et al. (1972) Phys. Rev. Lett., 29: 705-708; Maiti et al. (1997) Proc. Natl. Acad. Sci. USA, 94: 11753-1175). FCS measures the average diffusion rate of a fluorescent molecule within a small sample volume. The sample size can be as low as 10³ fluorescent molecules and the sample volume as low as the cytoplasm of a single bacterium. The diffusion rate is a function of the mass of the molecule and decreases as the mass increases. FCS can therefore be applied to protein-ligand interaction analysis by measuring the change in mass and therefore in diffusion rate of a molecule upon binding. In a typical experiment, the target to be analyzed is expressed as a recombinant protein with a sequence tag, such as a poly-histidine sequence, inserted at the N or C-terminus. The expression takes place in E. coli, yeast or insect cells. The protein is purified by chromatography. For example, the poly-histidine tag can be used to bind the expressed protein to a metal chelate column such as Ni2+ chelated on iminodiacetic acid agarose. The protein is then labeled with a fluorescent tag such as carboxytetramethylrhodamine or BODIPY® (Molecular Probes, Eugene, Oreg.). The protein is then exposed in solution to the potential ligand, and its diffusion rate is determined by FCS using instrumentation available from Carl Zeiss, Inc. (Thornwood, N.Y.). Ligand binding is determined by changes in the diffusion rate of the protein.

Surface-Enhanced Laser Desorption/Ionization (SELDI) was invented by Hutchens and Yip during the late 1980's (Hutchens and Yip (1993) Rapid Commun. Mass Spectrom. 7: 576-580). When coupled to a time-of-flight mass spectrometer (TOF), SELDI provides a mean to rapidly analyze molecules retained on a chip. It can be applied to ligand-protein interaction analysis by covalently binding the target protein on the chip and analyze by MS the small molecules that bind to this protein (Worrall et al. (1998) Anal. Biochem. 70: 750-756). In a typical experiment, the target to be analyzed is expressed as described for FCS. The purified protein is then used in the assay without further preparation. It is bound to the SELDI chip either by utilizing the poly-histidine tag or by other interaction such as ion exchange or hydrophobic interaction. The chip thus prepared is then exposed to the potential ligand via, for example, a delivery system capable to pipet the ligands in a sequential manner (autosampler). The chip is then submitted to washes of increasing stringency, for example a series of washes with buffer solutions containing an increasing ionic strength. After each wash, the bound material is analyzed by submitting the chip to SELDI-TOF. Ligands that specifically bind the target will be identified by the stringency of the wash needed to elute them.

Biacore relies on changes in the refractive index at the surface layer upon binding of a ligand to a protein immobilized on the layer. In this system, a collection of small ligands is injected sequentially in a 2-5 microliter cell with the immobilized protein. Binding is detected by surface plasmon resonance (SPR) by recording laser light refracting from the surface. In general, the refractive index change for a given change of mass concentration at the surface layer, is practically the same for all proteins and peptides, allowing a single method to be applicable for any protein (Liedberg et al. (1983) Sensors Actuators 4: 299-304; Malmquist (1993) Nature, 361: 186-187). In a typical experiment, the target to be analyzed is expressed as described for FCS. The purified protein is then used in the assay without further preparation. It is bound to the Biacore chip either by utilizing the poly-histidine tag or by other interaction such as ion exchange or hydrophobic interaction. The chip thus prepared is then exposed to the potential ligand via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics on rate and off rate allows the discrimination between non-specific and specific interaction.

IV. In vivo Inhibitor Assay

In one embodiment, a suspected herbicide, for example identified by in vitro screening, is applied to plants at various concentrations. The suspected herbicide is preferably sprayed on the plants. After application of the suspected herbicide, its effect on the plants, for example death or suppression of growth is recorded.

In another embodiment, an in vivo screening assay for inhibitors of the 1917, 2092, or 7724 activity uses transgenic plants, plant tissue, plant seeds or plant cells capable of overexpressing a nucleotide sequence having 1917, 2092, or 7724 activity, wherein the 1917, 2092, or 7724 gene product is enzymatically active in the transgenic plants, plant tissue, plant seeds or plant cells. The nucleotide sequence is preferably derived from an eukaryote, such as a yeast, but is preferably derived from a plant. In a further preferred embodiment, the nucleotide sequence is identical or substantially similar to the nucleotide sequence set forth in SEQ ID NO:1, or encodes an enzyme having 1917 activity, whose amino acid sequence is identical or substantially similar to the amino acid sequence set forth in SEQ ID NO:2. In a further preferred embodiment, the nucleotide sequence is identical or substantially similar to the nucleotide sequence set forth in SEQ ID NO:3, or encodes an enzyme having 2092 activity, whose amino acid sequence is identical or substantially similar to the amino acid sequence set forth in SEQ ID NO:4. In a further preferred embodiment, the nucleotide sequence is identical or substantially similar to the nucleotide sequence set forth in SEQ ID NO:5, or encodes an enzyme having 7724 activity, whose amino acid sequence is identical or substantially similar to the amino acid sequence set forth in SEQ ID NO:6. In another preferred embodiment, the nucleotide sequence is derived from a prokaryote, preferably a bacteria, e.g. E. coli.

A chemical is then applied to the transgenic plants, plant tissue, plant seeds or plant cells and to the isogenic non-transgenic plants, plant tissue, plant seeds or plant cells, and the growth or viability of the transgenic and non-transformed plants, plant tissue, plant seeds or plant cells are determined after application of the chemical and compared. Compounds capable of inhibiting the growth of the non-transgenic plants, but not affecting the growth of the transgenic plants are selected as specific inhibitors of 1917, 2092, or 7724 activity.

V. Herbicide Tolerant Plants

The present invention is further directed to plants, plant tissue, plant seeds, and plant cells tolerant to herbicides that inhibit the naturally occurring 1917, 2092, or 7724 activity in these plants, wherein the tolerance is conferred by an altered 1917, 2092, or 7724 activity. Altered 1917, 2092, or 7724 activity may be conferred upon a plant according to the invention by increasing expression of wild-type herbicide-sensitive 1917, 2092, or 7724 gene, for example by providing additional wild-type 1917, 2092, or 7724 genes and/or by overexpressing the endogenous 1917, 2092, or 7724 gene, for example by driving expression with a strong promoter. Altered 1917, 2092, or 7724 activity also may be accomplished by expressing nucleotide sequences that are substantially similar to SEQ ID NO: 1, SEQ ID NO:3, or SEQ ID NO:5, respectively, or homologs in a plant. Still further altered 1917, 2092, or 7724 activity is conferred on a plant by expressing modified herbicide-tolerant 1917, 2092, or 7724 genes in the plant. Combinations of these techniques may also be used. Representative plants include any plants to which these herbicides are applied for their normally intended purpose. Preferred are agronomically important crops such as cotton, soybean, oilseed rape, sugar beet, maize, rice, wheat, barley, oats, rye, sorghum, millet, turf, forage, turf grasses, and the like.

A. Increased Expression of Wild-Type 1917, 2092, or 7724

Achieving altered 1917, 2092, or 7724 activity through increased expression results in a level of 1917, 2092, or 7724 activity in the plant cell at least sufficient to overcome growth inhibition caused by the herbicide when applied in amounts sufficient to inhibit normal growth of control plants. The level of expressed enzyme generally is at least two times, preferably at least five times, and more preferably at least ten times the natively expressed amount. Increased expression may be due to multiple copies of a wild-type 1917, 2092, or 7724 gene; multiple occurrences of the coding sequence within the gene (i.e. gene amplification) or a mutation in the non-coding, regulatory sequence of the endogenous gene in the plant cell. Plants having such altered gene activity can be obtained by direct selection in plants by methods known in the art (see, e.g. U.S. Pat. Nos. 5,162,602, and 4,761,373, and references cited therein). These plants also may be obtained by genetic engineering techniques known in the art. Increased expression of a herbicide-sensitive 1917, 2092, or 7724 gene can also be accomplished by transforming a plant cell with a recombinant or chimeric DNA molecule comprising a promoter capable of driving expression of an associated structural gene in a plant cell operatively linked to a homologous or heterologous structural gene encoding the 1917, 2092, or 7724 protein or a homolog thereof. Preferably, the transformation is stable, thereby providing a heritable transgenic trait.

B. Expression of Modified Herbicide-Tolerant 1917, 2092, or 7724 Proteins

According to this embodiment, plants, plant tissue, plant seeds, or plant cells are stably transformed with a recombinant DNA molecule comprising a suitable promoter functional in plants operatively linked to a coding sequence encoding a herbicide tolerant form of the 1917, 2092, or 7724 protein. A herbicide tolerant form of the enzyme has at least one amino acid substitution, addition or deletion that confers tolerance to a herbicide that inhibits the unmodified, naturally occurring form of the enzyme. The transgenic plants, plant tissue, plant seeds, or plant cells thus created are then selected by conventional selection techniques, whereby herbicide tolerant lines are isolated, characterized, and developed. Below are described methods for obtaining genes that encode herbicide tolerant forms of 1917, 2092, or 7724 protein.

One general strategy involves direct or indirect mutagenesis procedures on microbes. For instance, a genetically manipulatable microbe such as E. coli or S. cerevisiae may be subjected to random mutagenesis in vivo with mutagens such as UV light or ethyl or methyl methane sulfonate. Mutagenesis procedures are described, for example, in Miller, Experiments in Molecular Genetics, Cold Spring Harbor Laboratory, Cold Spring Harbor, N.Y. (1972); Davis et al., Advanced Bacterial Genetics, Cold Spring Harbor Laboratory, Cold Spring Harbor, N.Y. (1980); Sherman et al., Methods in Yeast Genetics, Cold Spring Harbor Laboratory, Cold Spring Harbor, N.Y. (1983); and U.S. Pat. No. 4,975,374. The microbe selected for mutagenesis contains a normal, inhibitor-sensitive 1917, 2092, or 7724 gene and is dependent upon the activity conferred by this gene. The mutagenized cells are grown in the presence of the inhibitor at concentrations that inhibit the unmodified gene. Colonies of the mutagenized microbe that grow better than the unmutagenized microbe in the presence of the inhibitor (i.e. exhibit resistance to the inhibitor) are selected for further analysis. 1917, 2092, or 7724 genes conferring tolerance to the inhibitor are isolated from these colonies, either by cloning or by PCR amplification, and their sequences are elucidated. Sequences encoding altered gene products are then cloned back into the microbe to confirm their ability to confer inhibitor tolerance.

A method of obtaining mutant herbicide-tolerant alleles of a plant 1917, 2092, or 7724 gene involves direct selection in plants. For example, the effect of a mutagenized 1917, 2092, or 7724 gene on the growth inhibition of plants such as Arabidopsis, soybean, or maize is determined by plating seeds sterilized by art-recognized methods on plates on a simple minimal salts medium containing increasing concentrations of the inhibitor. Such concentrations are in the range of 0.001, 0.003, 0.01, 0.03, 0.1, 0.3, 1, 3, 10, 30, 110, 300, 1000 and 3000 parts per million (ppm). The lowest dose at which significant growth inhibition can be reproducibly detected is used for subsequent experiments. Determination of the lowest dose is routine in the art.

Mutagenesis of plant material is utilized to increase the frequency at which resistant alleles occur in the selected population. Mutagenized seed material is derived from a variety of sources, including chemical or physical mutagenesis or seeds, or chemical or physical mutagenesis or pollen (Neuffer, In Maize for Biological Research Sheridan, ed. Univ. Press, Grand Forks, N.Dak., pp. 61-64 (1982)), which is then used to fertilize plants and the resulting M₁ mutant seeds collected. Typically for Arabidopsis, M₂ seeds (Lehle Seeds, Tucson, Ariz.), which are progeny seeds of plants grown from seeds mutagenized with chemicals, such as ethyl methane sulfonate, or with physical agents, such as gamma rays or fast neutrons, are plated at densities of up to 10,000 seeds/plate (10 cm diameter) on minimal salts medium containing an appropriate concentration of inhibitor to select for tolerance. Seedlings that continue to grow and remain green 7-21 days after plating are transplanted to soil and grown to maturity and seed set. Progeny of these seeds are tested for tolerance to the herbicide. If the tolerance trait is dominant, plants whose seed segregate 3:1/resistant:sensitive are presumed to have been heterozygous for the resistance at the M₂ generation. Plants that give rise to all resistant seed are presumed to have been homozygous for the resistance at the M₂ generation. Such mutagenesis on intact seeds and screening of their M₂ progeny seed can also be carried out on other species, for instance soybean (see, e.g. U.S. Pat. No. 5,084,082). Alternatively, mutant seeds to be screened for herbicide tolerance are obtained as a result of fertilization with pollen mutagenized by chemical or physical means.

Confirmation that the genetic basis of the herbicide tolerance is a 1917, 2092, or 7724 gene is ascertained as exemplified below. First, alleles of the 1917, 2092, or 7724 gene from plants exhibiting resistance to the inhibitor are isolated using PCR with primers based either upon the Arabidopsis cDNA coding sequences shown in SEQ ID NO:1, SEQ ID NO:3, or SEQ ID NO:5, respectively, or, more preferably, based upon the unaltered 1917, 2092, or 7724 gene sequence from the plant used to generate tolerant alleles. After sequencing the alleles to determine the presence of mutations in the coding sequence, the alleles are tested for their ability to confer tolerance to the inhibitor on plants into which the putative tolerance-conferring alleles have been transformed. These plants can be either Arabidopsis plants or any other plant whose growth is susceptible to the 1917, 2092, or 7724 inhibitors. Second, the inserted 1917, 2092, or 7724 genes are mapped relative to known restriction fragment length polymorphisms (RFLPs) (See, for example, Chang et al. Proc. Natl. Acad, Sci, USA 85: 6856-6860 (1988); Nam et al., Plant Cell 1: 699-705 (1989), cleaved amplified polymorphic sequences (CAPS) (Konieczny and Ausubel (1993) The Plant Journal, 4(2): 403-410), or SSLPs (Bell and Ecker (1994) Genomics, 19: 137-144). The 1917, 2092, or 7724 inhibitor tolerance trait is independently mapped using the same markers. When tolerance is due to a mutation in that 1917, 2092, or 7724 gene, the tolerance trait maps to a position indistinguishable from the position of the 1917, 2092, or 7724 gene.

Another method of obtaining herbicide-tolerant alleles of a 1917, 2092, or 7724 gene is by selection in plant cell cultures. Explants of plant tissue, e.g. embryos, leaf disks, etc. or actively growing callus or suspension cultures of a plant of interest are grown on medium in the presence of increasing concentrations of the inhibitory herbicide or an analogous inhibitor suitable for use in a laboratory environment. Varying degrees of growth are recorded in different cultures. In certain cultures, fast-growing variant colonies arise that continue to grow even in the presence of normally inhibitory concentrations of inhibitor. The frequency with which such faster-growing variants occur can be increased by treatment with a chemical or physical mutagen before exposing the tissues or cells to the inhibitor. Putative tolerance-conferring alleles of the 1917, 2092, or 7724 gene are isolated and tested as described in the foregoing paragraphs. Those alleles identified as conferring herbicide tolerance may then be engineered for optimal expression and transformed into the plant. Alternatively, plants can be regenerated from the tissue or cell cultures containing these alleles.

Still another method involves mutagenesis of wild-type, herbicide sensitive plant 1917, 2092, or 7724 genes in bacteria or yeast, followed by culturing the microbe on medium that contains inhibitory concentrations (i.e. sufficient to cause abnormal growth, inhibit growth or cause cell death) of the inhibitor, and then selecting those colonies that grow normally in the presence of the inhibitor. More specifically, a plant cDNA, such as the Arabidopsis cDNA encoding the 1917, 2092, or 7724 protein, is cloned into a microbe that otherwise lacks the 1917, 2092, or 7724 activity. The transformed microbe is then subjected to in vivo mutagenesis or to in vitro mutagenesis by any of several chemical or enzymatic methods known in the art, e.g. sodium bisulfite (Shortle et al., Methods Enzymol. 100:457-468 (1983); methoxylamine (Kadonaga et al., Nucleic Acids Res. 13:1733-1745 (1985); oligonucleotide-directed saturation mutagenesis (Hutchinson et al., Proc. Natl. Acad. Sci. USA, 83:710-714 (1986); or various polymerase misincorporation strategies (see, e.g. Shortle et al., Proc. Natl. Acad. Sci. USA, 79:1588-1592 (1982); Shiraishi et al., Gene 64:313-319 (1988); and Leung et al., Technique 1:11-15 (1989). Colonies that grow normally in the presence of normally inhibitory concentrations of inhibitor are picked and purified by repeated restreaking. Their plasmids are purified and tested for the ability to confer tolerance to the inhibitor by retransforming them into the microbe lacking 1917, 2092, or 7724 activity. The DNA sequences of cDNA inserts from plasmids that pass this test are then determined.

Herbicide resistant 1917, 2092, or 7724 proteins are also obtained using methods involving in vitro recombination, also called DNA shuffling. By DNA shuffling, mutations, preferably random mutations, are introduced into nucleotide sequences encoding 1917, 2092, or 7724 activity. DNA shuffling also leads to the recombination and rearrangement of sequences within a 1917, 2092, or 7724 gene or to recombination and exchange of sequences between two or more different of 1917, 2092, or 7724 genes. These methods allow for the production of millions of mutated 1917, 2092, or 7724 coding sequences. The mutated genes, or shuffled genes, are screened for desirable properties, e.g. improved tolerance to herbicides and for mutations that provide broad-spectrum tolerance to the different classes of inhibitor chemistry. Such screens are well within the skills of a routineer in the art.

In a preferred embodiment, a mutagenized 1917, 2092, or 7724 gene is formed from at least one template 1917, 2092, or 7724 gene, wherein the template 1917, 2092, or 7724 gene has been cleaved into double-stranded random fragments of a desired size, and comprising the steps of adding to the resultant population of double-stranded random fragments one or more single or double-stranded oligonucleotides, wherein said oligonucleotides comprise an area of identity and an area of heterology to the double-stranded random fragments; denaturing the resultant mixture of double-stranded random fragments and oligonucleotides into single-stranded fragments; incubating the resultant population of single-stranded fragments with a polymerase under conditions which result in the annealing of said single-stranded fragments at said areas of identity to form pairs of annealed fragments, said areas of identity being sufficient for one member of a pair to prime replication of the other, thereby forming a mutagenized double-stranded polynucleotide; and repeating the second and third steps for at least two further cycles, wherein the resultant mixture in the second step of a further cycle includes the mutagenized double-stranded polynucleotide from the third step of the previous cycle, and the further cycle forms a further mutagenized double-stranded polynucleotide, wherein the mutagenized polynucleotide is a mutated 1917, 2092, or 7724 gene having enhanced tolerance to a herbicide which inhibits naturally occurring 1917, 2092, or 7724 activity. In a preferred embodiment, the concentration of a single species of double-stranded random fragment in the population of double-stranded random fragments is less than 1% by weight of the total DNA. In a further preferred embodiment, the template double-stranded polynucleotide comprises at least about 100 species of polynucleotides. In another preferred embodiment, the size of the double-stranded random fragments is from about 5 bp to 5 kb. In a further preferred embodiment, the fourth step of the method comprises repeating the second and the third steps for at least 10 cycles. Such method is described e.g. in Stemmer et al. (1994) Nature 370: 389-391, in U.S. Pat. Nos. 5,605,793, 5,811,238 and in Crameri et al. (1998) Nature 391: 288-291, as well as in WO 97/20078, and these references are incorporated herein by reference.

In another preferred embodiment, any combination of two or more different 1917, 2092, or 7724 genes are mutagenized in vitro by a staggered extension process (StEP), as described e.g. in Zhao et al. (1998) Nature Biotechnology 16: 258-261. The two or more 1917, 2092, or 7724 genes are used as template for PCR amplification with the extension cycles of the PCR reaction preferably carried out at a lower temperature than the optimal polymerization temperature of the polymerase. For example, when a thermostable polymerase with an optimal temperature of approximately 72° C. is used, the temperature for the extension reaction is desirably below 72° C., more desirably below 65° C., preferably below 60° C., more preferably the temperature for the extension reaction is 55° C. Additionally, the duration of the extension reaction of the PCR cycles is desirably shorter than usually carried out in the art, more desirably it is less than 30 seconds, preferably it is less than 15 seconds, more preferably the duration of the extension reaction is 5 seconds. Only a short DNA fragment is polymerized in each extension reaction, allowing template switch of the extension products between the starting DNA molecules after each cycle of denaturation and annealing, thereby generating diversity among the extension products. The optimal number of cycles in the PCR reaction depends on the length of the 1917, 2092, or 7724 genes to be mutagenized but desirably over 40 cycles, more desirably over 60 cycles, preferably over 80 cycles are used. Optimal extension conditions and the optimal number of PCR cycles for every combination of 1917, 2092, or 7724 genes are determined as described in using procedures well-known in the art. The other parameters for the PCR reaction are essentially the same as commonly used in the art. The primers for the amplification reaction are preferably designed to anneal to DNA sequences located outside of the 1917, 2092, or 7724 genes, e.g. to DNA sequences of a vector comprising the 1917, 2092, or 7724 genes, whereby the different 1917, 2092, or 7724 genes used in the PCR reaction are preferably comprised in separate vectors. The primers desirably anneal to sequences located less than 500 bp away from 1917, 2092, or 7724 sequences, preferably less than 200 bp away from the 1917, 2092, or 7724 sequences, more preferably less than 120 bp away from the 1917, 2092, or 7724 sequences. Preferably, the 1917, 2092, or 7724 sequences are surrounded by restriction sites, which are included in the DNA sequence amplified during the PCR reaction, thereby facilitating the cloning of the amplified products into a suitable vector.

In another preferred embodiment, fragments of 1917, 2092, or 7724 genes having cohesive ends are produced as described in WO 98/05765. The cohesive ends are produced by ligating a first oligonucleotide corresponding to a part of a 1917, 2092, or 7724 gene to a second oligonucleotide not present in the gene or corresponding to a part of the gene not adjoining to the part of the gene corresponding to the first oligonucleotide, wherein the second oligonucleotide contains at least one ribonucleotide. A double-stranded DNA is produced using the first oligonucleotide as template and the second oligonucleotide as primer. The ribonucleotide is cleaved and removed. The nucleotide(s) located 5′ to the ribonucleotide is also removed, resulting in double-stranded fragments having cohesive ends. Such fragments are randomly reassembled by ligation to obtain novel combinations of gene sequences.

In yet another embodiment, herbicide-resistant 1917, 2092, or 7724 proteins are produced using the incremental truncation for the creation of hybrid enzymes (ITCHY), as described in Ostermeier et al. (1999) Nature Biotechnology 17:1205-1209), and this reference is incorporated herein by reference.

Any 1917, 2092, or 7724 gene or any combination of 1917, 2092, or 7724 genes is used for in vitro recombination in the context of the present invention, for example, a 1917, 2092, or 7724 gene derived from a plant, such as, e.g. Arabidopsis thaliana, e.g. a 1917, 2092, or 7724 gene set forth in SEQ ID NO:1, SEQ ID NO:3, or SEQ ID NO:5, respectively. A 1917-like gene from human (Girjes et al. (1995) Gene, 164: 347-350), a 2092-like gene from human (Shiba et al. (1995) Biochemistry, 33: 10340-10349), a 7724-like gene from yeast (Culver et al. (1997) J. Biol. Chemistry, 272: 13203-13210), all of which are incorporated herein by reference. Whole 1917, 2092, or 7724 genes or portions thereof are used in the context of the present invention. The library of mutated 1917, 2092, or 7724 genes obtained by the methods described above are cloned into appropriate expression vectors and the resulting vectors are transformed into an appropriate host, for example an algae like Chlamydomonas, a yeast or a bacteria. An appropriate host is preferably a host that otherwise lacks 1917, 2092, or 7724 activity, for example E. coli. Host cells transformed with the vectors comprising the library of mutated 1917, 2092, or 7724 genes are cultured on medium that contains inhibitory concentrations of the inhibitor and those colonies that grow in the presence of the inhibitor are selected. Colonies that grow in the presence of normally inhibitory concentrations of inhibitor are picked and purified by repeated restreaking. Their plasmids are purified and the DNA sequences of cDNA inserts from plasmids that pass this test are then determined.

An assay for identifying a modified 1917, 2092, or 7724 gene that is tolerant to an inhibitor may be performed in the same manner as the assay to identify inhibitors of the 1917, 2092, or 7724 activity (Inhibitor Assay, above) with the following modifications: First, a mutant 1917, 2092, or 7724 protein is substituted in one of the reaction mixtures for the wild-type 1917, 2092, or 7724 protein of the inhibitor assay. Second, an inhibitor of wild-type enzyme is present in both reaction mixtures. Third, mutated activity (activity in the presence of inhibitor and mutated enzyme) and unmutated activity (activity in the presence of inhibitor and wild-type enzyme) are compared to determine whether a significant increase in enzymatic activity is observed in the mutated activity when compared to the unmutated activity. Mutated activity is any measure of activity of the mutated enzyme while in the presence of a suitable substrate and the inhibitor. Unmutated activity is any measure of activity of the wild-type enzyme while in the presence of a suitable substrate and the inhibitor.

In addition to being used to create herbicide-tolerant plants, genes encoding herbicide tolerant 1917, 2092, or 7724 protein can also be used as selectable markers in plant cell transformation methods. For example, plants, plant tissue, plant seeds, or plant cells transformed with a heterologous DNA sequence can also be transformed with a sequence encoding an altered 1917, 2092, or 7724 activity capable of being expressed by the plant. The transformed cells are transferred to medium containing an inhibitor of the enzyme in an amount sufficient to inhibit the growth or survivability of plant cells not expressing the modified coding sequence, wherein only the transformed cells will grow. The method is applicable to any plant cell capable of being transformed with a modified 1917, 2092, or 7724 gene, and can be used with any heterologous DNA sequence of interest. Expression of the heterologous DNA sequence and the modified gene can be driven by the same promoter functional in plant cells, or by separate promoters.

VI. Plant Transformation Technology

A wild-type or herbicide-tolerant form of the 1917, 2092, or 7724 gene, or homologs thereof, can be incorporated in plant or bacterial cells using conventional recombinant DNA technology. Generally, this involves inserting a DNA molecule encoding the 1917, 2092, or 7724 gene into an expression system to which the DNA molecule is heterologous (i.e., not normally present) using standard cloning procedures known in the art. The vector contains the necessary elements for the transcription and translation of the inserted protein-coding sequences in a host cell containing the vector. A large number of vector systems known in the art can be used, such as plasmids, bacteriophage viruses and other modified viruses. The components of the expression system may also be modified to increase expression. For example, truncated sequences, nucleotide substitutions, nucleotide optimization or other modifications may be employed. Expression systems known in the art can be used to transform virtually any crop plant cell under suitable conditions. A heterologous DNA sequence comprising a wild-type or herbicide-tolerant form of the 1917, 2092, or 7724 gene is preferably stably transformed and integrated into the genome of the host cells. In another preferred embodiment, the heterologous DNA sequence comprising a wild-type or herbicide-tolerant form of the 1917, 2092, or 7724 gene located on a self-replicating vector. Examples of self-replicating vectors are viruses, in particular gemini viruses. Transformed cells can be regenerated into whole plants such that the chosen form of the 1917, 2092, or 7724 gene confers herbicide tolerance in the transgenic plants.

A. Requirements for Construction of Plant Expression Cassettes

Gene sequences intended for expression in transgenic plants is first assembled in expression cassettes behind a suitable promoter expressible in plants. The expression cassettes may also comprise any further sequences required or selected for the expression of the heterologous DNA sequence. Such sequences include, but are not restricted to, transcription terminators, extraneous sequences to enhance expression such as introns, vital sequences, and sequences intended for the targeting of the gene product to specific organelles and cell compartments. These expression cassettes can then be easily transferred to the plant transformation vectors described infra. The following is a description of various components of typical expression cassettes.

1. Promoters

The selection of the promoter used in expression cassettes will determine the spatial and temporal expression pattern of the heterologous DNA sequence in the plant transformed with this DNA sequence. Selected promoters will express heterologous DNA sequences in specific cell types (such as leaf epidermal cells, mesophyll cells, root cortex cells) or in specific tissues or organs (roots, leaves or flowers, for example) and the selection will reflect the desired location of accumulation of the gene product. Alternatively, the selected promoter may drive expression of the gene under various inducing conditions. Promoters vary in their strength, i.e., ability to promote transcription. Depending upon the host cell system utilized, any one of a number of suitable promoters known in the art can be used. For example, for constitutive expression, the CaMV 35S promoter, the rice actin promoter, or the ubiquitin promoter may be used. For regulatable expression, the chemically inducible PR-1 promoter from tobacco or Arabidopsis may be used (see, e.g., U.S. Pat. No. 5,689,044).

2. Transcriptional Terminators

A variety of transcriptional terminators are available for use in expression cassettes. These are responsible for the termination of transcription beyond the heterologous DNA sequence and its correct polyadenylation. Appropriate transcriptional terminators are those that are known to function in plants and include the CaMV 35S terminator, the tml terminator, the nopaline synthase terminator and the pea rbcS E9 terminator. These can be used in both monocotyledonous and dicotyledonous plants.

3. Sequences for the Enhancement or Regulation of Expression

Numerous sequences have been found to enhance gene expression from within the transcriptional unit and these sequences can be used in conjunction with the genes of this invention to increase their expression in transgenic plants. For example, various intron sequences such as introns of the maize AdhI gene have been shown to enhance expression, particularly in monocotyledonous cells. In addition, a number of non-translated leader sequences derived from viruses are also known to enhance expression, and these are particularly effective in dicotyledonous cells.

4. Coding Sequence Optimization

The coding sequence of the selected gene may be genetically engineered by altering the coding sequence for optimal expression in the crop species of interest. Methods for modifying coding sequences to achieve optimal expression in a particular crop species are well known (see, e.g. Perlak et al., Proc. Natl. Acad. Sci. USA 88: 3324 (1991); and Koziel et al., Bio/technol. 11: 194 (1993)).

5. Targeting of the Gene Product Within the Cell

Various mechanisms for targeting gene products are known to exist in plants and the sequences controlling the functioning of these mechanisms have been characterized in some detail. For example, the targeting of gene products to the chloroplast is controlled by a signal sequence found at the amino terminal end of various proteins which is cleaved during chloroplast import to yield the mature protein (e.g. Comai et al. J. Biol. Chem. 263: 15104-15109 (1988)). Other gene products are localized to other organelles such as the mitochondrion and the peroxisome (e.g. Unger et al. Plant Molec. Biol. 13: 411-418 (1989)). The cDNAs encoding these products can also be manipulated to effect the targeting of heterologous products encoded by DNA sequences to these organelles. In addition, sequences have been characterized which cause the targeting of products encoded by DNA sequences to other cell compartments. Amino terminal sequences are responsible for targeting to the ER, the apoplast, and extracellular secretion from aleurone cells (Koehler & Ho, Plant Cell 2: 769-783 (1990)). Additionally, amino terminal sequences in conjunction with carboxy terminal sequences are responsible for vacuolar targeting of gene products (Shinshi et al. Plant Molec. Biol. 14: 357-368 (1990)). By the fusion of the appropriate targeting sequences described above to heterologous DNA sequences of interest it is possible to direct this product to any organelle or cell compartment.

B. Construction of Plant Transformation Vectors

Numerous transformation vectors available for plant transformation are known to those of ordinary skill in the plant transformation arts, and the genes pertinent to this invention can be used in conjunction with any such vectors. The selection of vector will depend upon the preferred transformation technique and the target species for transformation. For certain target species, different antibiotic or herbicide selection markers may be preferred. Selection markers used routinely in transformation include the nptII gene, which confers resistance to kanamycin and related antibiotics (Messing & Vierra. Gene 19: 259-268 (1982); Bevan et al., Nature 304:184-187 (1983)), the bar gene, which confers resistance to the herbicide phosphinothricin (White et al., Nucl. Acids Res 18: 1062 (1990), Spencer et al. Theor. Appl. Genet 79: 625-631 (1990)), the hph gene, which confers resistance to the antibiotic hygromycin (Blochinger & Diggelmann, Mol Cell Biol 4: 2929-2931), the manA gene, which allows for positive selection in the presence of mannose (Miles and Guest (1984) Gene, 32:41-48; U.S. Pat. No. 5,767,378), and the dhfr gene, which confers resistance to methotrexate (Bourouis et al., EMBO J. 2(7): 1099-1104 (1983)), and the EPSPS gene, which confers resistance to glyphosate (U.S. Pat. Nos. 4,940,935 and 5,188,642).

1. Vectors Suitable for Agrobacterium Transformation

Many vectors are available for transformation using Agrobacterium tumefaciens. These typically carry at least one T-DNA border sequence and include vectors such as pBIN19 (Bevan, Nucl. Acids Res. (1984)). Typical vectors suitable for Agrobacterium transformation include the binary vectors pCIB200 and pCIB2001, as well as the binary vector pCIB10 and hygromycin selection derivatives thereof. (See, for example, U.S. Pat. No. 5,639,949).

2. Vectors Suitable for non-Agrobacterium Transformation

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. Typical vectors suitable for non-Agrobacterium transformation include pCIB3064, pSOG19, and pSOG35. (See, for example, U.S. Pat. No. 5,639,949).

C. Transformation Techniques

Once the coding sequence of interest has been cloned into an expression system, it is transformed into a plant cell. Methods for transformation and regeneration of plants are well known in the art. For example, Ti plasmid vectors have been utilized for the delivery of foreign DNA, as well as direct DNA uptake, liposomes, electroporation, micro-injection, and microprojectiles. In addition, bacteria from the genus Agrobacterium can be utilized to transform plant cells.

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG- or electroporation-mediated uptake, particle bombardment-mediated delivery, or microinjection. In each case the transformed cells are regenerated to whole plants using standard techniques known in the art.

Transformation of most monocotyledon species has now also become routine. Preferred techniques include direct gene transfer into protoplasts using PEG or electroporation techniques, particle bombardment into callus tissue, as well as Agrobacterium-mediated transformation.

D. Plastid Transformation

In another preferred embodiment, a nucleotide sequence encoding a polypeptide having 1917, 2092, or 7724 activity is directly transformed into the plastid genome. Plastid expression, in which genes are inserted by homologous recombination into the several thousand copies of the circular plastid genome present in each plant cell, takes advantage of the enormous copy number advantage over nuclear-expressed genes to permit expression levels that can readily exceed 10% of the total soluble plant protein. In a preferred embodiment, the nucleotide sequence is inserted into a plastid-targeting vector and transformed into the plastid genome of a desired plant host. Plants homoplasmic for plastid genomes containing the nucleotide sequence are obtained, and are preferentially capable of high expression of the nucleotide sequence.

Plastid transformation technology is for example extensively described in U.S. Pat. Nos. 5,451,513, 5,545,817, 5,545,818, and 5,877,462 in PCT application no. WO 95/16783 and WO 97/32977, and in McBride et al. (1994) Proc. Natl. Acad. Sci. USA 91, 7301-7305, all incorporated herein by reference in their entirety. The basic technique for plastid transformation involves introducing regions of cloned plastid DNA flanking a selectable marker together with the nucleotide sequence into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rpsl2 genes conferring resistance to spectinomycin and/or streptomycin are utilized as selectable markers for transformation (Svab, Z., Hajdukiewicz, P., and Maliga, P. (1990) Proc. Natl. Acad. Sci. USA 87, 8526-8530; Staub, J. M., and Maliga, P. (1992) Plant Cell 4, 39-45). The presence of cloning sites between these markers allowed creation of a plastid targeting vector for introduction of foreign genes (Staub, J. M., and Maliga, P. (1993) EMBO J. 12, 601-606). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab, Z., and Maliga, P. (1993) Proc. Natl. Acad. Sci. USA 90, 913-917). Other selectable markers useful for plastid transformation are known in the art and encompassed within the scope of the invention.

VII. Breeding

The wild-type or altered form of a 1917, 2092, or 7724 gene of the present invention can be utilized to confer herbicide tolerance to a wide variety of plant cells, including those of gymnosperms, monocots, and dicots. Although the gene can be inserted into any plant cell falling within these broad classes, it is particularly useful in crop plant cells, such as rice, wheat, barley, rye, corn, potato, carrot, sweet potato, sugar beet, bean, pea, chicory, lettuce, cabbage, cauliflower, broccoli, turnip, radish, spinach, asparagus, onion, garlic, eggplant, pepper, celery, carrot, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum and sugarcane.

The high-level expression of a wild-type 1917, 2092, or 7724 gene and/or the expression of herbicide-tolerant forms of a 1917, 2092, or 7724 gene conferring herbicide tolerance in plants, in combination with other characteristics important for production and quality, can be incorporated into plant lines through breeding approaches and techniques known in the art.

Where a herbicide tolerant 1917, 2092, or 7724 gene allele is obtained by direct selection in a crop plant or plant cell culture from which a crop plant can be regenerated, it is moved into commercial varieties using traditional breeding techniques to develop a herbicide tolerant crop without the need for genetically engineering the allele and transforming it into the plant.

The invention will be further described by reference to the following detailed examples. These examples are provided for purposes of illustration only, and are not intended to be limiting unless otherwise specified.

### EXAMPLES

Standard recombinant DNA and molecular cloning techniques used here are well known in the art and are described by J. Sambrook, et al., Molecular Cloning: A Laboratory Manual, 3d Ed., Cold Spring Harbor, N.Y.: Cold Spring Harbor Laboratory Press (2001); by T. J. Silhavy, M. L. Berman, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, New York, John Wiley and Sons Inc., (1988), Reiter, et al., Methods in Arabidopsis Research, World Scientific Press (1992), Schultz et al., Plant Molecular Biology Manual, Kluwer Academic Publishers (1998), and Reiter, et al., Methods in Arabidopsis Research, World Scientific Press (1992). These references describe the standard techniques used for all steps in tagging and cloning genes from T-DNA mutagenized populations of Arabidopsis: plant infection and transformation; screening for the identification of seedling mutants; cosegregation analysis; and plasmid rescue.

### Example 1

Plant Infection and Transformation in Tagged Embryo-Lethal Lines 1917, 2092, and 7724

Arabidopsis plants (strain Columbia) are inverted, and their leaves are vacuum-infiltrated with Agrobacterium (1X dilution of Agrobacterium grown to OD600 of 0.8 in 10 MM MgCl₂). T1 seed is collected from these plants, and germinated on an agar-solidified medium containing (50 ug/ml Basta) or sprayed in soil (400 μg/ml Basta). Typically, 0.1% to 1.0% of the plants contain T-DNA inserts in a population of T1 transformants. Furthermore, the plants that survive on Basta selection are hemizygous for the T-DNA insertion and thus the Basta selectable marker.

Mutants blocked in growth or development are identified by examining T2 progeny using an embryo screen and recovering those plants that contained 25% aborted seeds. Using segregation analysis of T2 individuals, approximately one-third of the mutants are tagged.

### Example 2

Embryo Screen for the Identification of Mutants Blocked in Early Development from Tagged Embryo-Lethal Lines 1917, 2092, and 7724

Essential genes are identified through the isolation of lethal mutants blocked in early development. Examples of lethal mutants include those blocked in the formation of the male or female gametes, embryo, or resulting seedling. Gametophytic mutants are found by examining T1 insertion lines for the presence of 50% aborted pollen grains or ovules. Embryo defective lethal mutants produce 25% defective seeds following self-pollination of T1 plants (see Errampalli et al. 1991, Plant Cell 3:149-157; Castle et al. 1993, Mol Gen Genet 241:504-514). Seedling lethal mutants segregate for 25% seedlings that exhibit a lethal phenotype.

The T1 line #1917 shows 25% defective seeds that contain embryos that are arrested at the globular stage of development.

The T1 line #2092 shows 25% defective seeds that contain embryos that are arrested at the preglobular to globular stages of development.

The T1 line #7724 shows 25% defective seeds that contain embryos that are arrested at the torpedo to cotyledon stage of development.

### Example 3

Cosegregation Analysis for Tagged Embryo-Lethal Lines 1917, 2092, and 7724

The linkage of the mutation to the T-DNA insert is established after identifying a transformed line segregating for a lethal phenotype of interest. A line segregating with a single functional insert will segregate for resistance in the ratio of 2:1 (resistance:sensitive) to the selectable marker Basta. In this case, one-quarter of the T2 progeny will fail to germinate due to embryo lethality, resulting in a reduction of the normal 3:1 ratio to 2:1. Each of the Basta resistant progeny are therefore heterozygous for the mutation if the T-DNA insert is causing the mutant phenotype. To confirm cosegregation of the T-DNA and the mutant phenotype, Basta resistant progeny are transplanted to soil and screened again for the presence of 25% aborted seeds.

For 1917, each of the 18 progeny examined contains approximately 25% aborted seeds with the expected phenotype. These results confirm that there is no evidence for recombination between the T-DNA and the mutation. Single plant southern blot analysis suggests that the T-DNA insertion in line #1917 consists of a simple insertion.

For 2092, each of the 35 progeny examined contains approximately 25% aborted seeds with the expected phenotype. These results confirm that there is no evidence for recombination between the T-DNA and the mutation. Single plant Southern blot analysis suggests that the insertion in line #2092 consists of a at least three tandem T-DNA elements. Cosegregation analysis shows that hygromycin resistance and the mutant phenotype in line 2092 exhibit complete linkage in 35 selfed progeny from a selfed heterozygote.

For 7724, each of the 37 progeny examined contains approximately 25% aborted seeds with the expected phenotype. These results confirm that there is no evidence for recombination between the T-DNA and the mutation. Cosegregation analysis shows that Basta resistance and the mutant phenotype in line 7724 exhibit complete linkage in 37 selfed progeny from a selfed heterozygote.

### Example 4a

Plasmid Rescue from Tagged Embryo-Lethal Line 1917

Arabidopsis genomic DNA is isolated as described Reiter et al in Methods in Arabidopsis Research, World Scientific Press (1992). Genomic DNA is digested with a restriction endonuclease and ligated overnight. After ligation, the DNA is transformed into competent E. coli strain XL-1 Blue, DH10B, DH5 alpha, or the like, and colonies are selected on semi-solid medium containing ampicillin. Resistant colonies are picked into liquid medium with ampicillin and grown overnight. Plasmid DNA is isolated and digested with the rescue enzyme and analyzed on agarose gels containing ethidium bromide for visualization. Plasmids that represent different size classes are sequenced using primers that flank the plant DNA portion of the rescue element and the sequence is analyzed to determine what portion is plant DNA and what gene has been disrupted.

One method of confirming that the disrupted gene is the cause of the mutant phenotype is to transform a wild-type form of the gene into the mutant plant. Alternatively, the mutant is phenocopied by specifically reducing expression of the disrupted gene in transgenic plants expressing an antisense version of the gene behind a synthetic promoter (Guyer et al. (1998) Genetics, 149: 633-639).

### Example 4b

Plasmid Rescue from Tagged Embryo-Lethal Line 2092

Arabidopsis genomic DNA is isolated as described in Reiter et al in Methods in Arabidopsis Research, World Scientific Press (1992). Genomic DNA is digested with a restriction endonuclease and ligated overnight. After ligation, the DNA is transformed into competent E. coli strain XL-1 Blue, DH10B, DH5 alpha, or the like, and colonies are selected on semi-solid medium containing ampicillin. Resistant colonies are picked into liquid medium with ampicillin and grown overnight. Plasmid DNA is isolated and digested with the rescue enzyme and analyzed on agarose gels containing ethidium bromide for visualization. Plasmids that represent different size classes are sequenced using primers that flank the plant DNA portion of the rescue element and the sequence is analyzed to determine what portion is plant DNA and what gene has been disrupted.

One method of confirming that the disrupted gene is the cause of the mutant phenotype is to transform a wild-type form of the gene into the mutant plant. Alternatively, the mutant is phenocopied by specifically reducing expression of the disrupted gene in transgenic plants expressing an antisense version of the gene behind a synthetic promoter (Guyer et al. (1998) Genetics, 149: 633-639).

### Example 4c

Border Rescue from Tagged Embryo-Lethal Line 7724

Arabidopsis genomic DNA is isolated as described in Reiter et al in Methods in Arabidopsis Research, World Scientific Press (1992). DNA flanking the borders of line #7724 is isolated using TAIL PCR. A series of 12 TAIL PCR reactions are performed on DNA from line #7724; 6 arbitrary degenerate primers (CA50 primer: 5′ NGT CGA SWG ANA WGA A 3′: SEQ ID NO:9 (128-fold, AD2 from Liu et al. (1995) The Plant Journal, 8: 457-463); CA51 primer: 5′ TGW GNA GSA NCA SAG A 3′: SEQ ID NO:10 (128-fold derivative of AD1 from Liu and Whittier (1995) Genomics, 25: 674-681); CA52 primer: 5′ AGW GNA GWA NCA WAG G 3′: SEQ ID NO:11 (128-fold, AD2 from Liu and Whittier (1995) Genomics, 25:674-681); CA53 primer: 5′ STT GNT AST NCT NTG C 3′: SEQ ID NO:12 (256-fold, AD5 from Tsugeki et al. (1996) The Plant Journal, 10: 479-489); CA54 primer: 5′ NTC GAS TWT SGW GTT 3′: SEQ ID NO:13 (64-fold, ADI from Liu et al. (1995) The Plant Journal, 8: 457-463); and CA55 primer: 5′ WGT GNA GWA NCA NAG A 3′: SEQ ID NO:14 (256-fold, AD3 from Liu et al. (1995) The Plant Journal, 8: 457-463) are used in combination with two sets of nested, and T-DNA specific primers for the right border (CA66 primer: 5′ ATT AGG CAC CCC AGG CTT TAC ACT TTA TG 3′: SEQ ID NO:15 (pCSA104 right border primary primer); CA67 primer: 5′ GTA TGT TGT GTG GAA TTG TGA GCG GAT AAC 3′: SEQ ID NO:16 (pCSA104 right border secondary primer); and CA68 primer: 5′ TAA CAA TTT CAC ACA GGA AAC AGC TAT GAC 3′: SEQ ID NO:17 (pCSA104 right border tertiary primer) as well as for the left border (JM33 primer: 5′ TAG CAT CTG AAT TTC ATA ACC AAT CTC GAT ACA C 3′: SEQ ID NO:18 (pCSA104 left border tertiary primer; JM34 primer: 5′ GCT TCC TAT TAT ATC TTC CCA AAT TAC CAA TAC A 3′: SEQ ID NO:19 (pCSA104 left border secondary primer); and JM35 primer:

5′ GCC TTT TCA GAA ATG GAT AAA TAG CCT TGC TTC C 3′: SEQ ID NO:20 (pCSA104 left border primary primer) of the T-DNA region of pCSA104.

A total of 10 products are obtained from the left border, two of the sequenced products represent both sides of the T-DNA insertion. PCR primers specific to the genomic region are then designed and used to confirm the border products obtained by TAIL PCR.

### Example 5a

Sequence Analysis of Tagged Embryo-Lethal Line #1917 From the Insertional Mutant Collection

Analysis of Arabidopsis thaliana genomic DNA sequence flanking the right border region of the T-DNA insert in line 1917 reveals a single exon open reading frame of 1,656 bp (SEQ ID NO:1). Arabidopsis thaliana genomic DNA flanking the T-DNA border is identical to the ESTs 166E6T7 (Genbank Accession #R30603) and 203E14T7 (Genbank Accession # H77096) and to portions of the genomic survey sequences T19C17TR (Genbank Accession # B28763) F13K23-Sp6 (Genbank Accession # B 10372).

Using GAP (SeqWeb version 10.0, GCG), pairwise comparisons of the protein sequence (SEQ ID NO:2) and input sequences shown below give a measure of similarity between SEQ ID NO:2 and the indicated sequences, and they are summarized below.

| GenPept Accession # | % Identity | % Similarity |
|---|---|---|
| P37880¹ | 47 | 63 |
| NP_002878.1² | 46 | 63 |
| Q55486³ | 48 | 62 |
| Q19825⁴ | 43 | 60 |
| AE001641⁵ | 42 | 57 |
| AL079345⁶ | 40 | 57 |
| P43832⁷ | 40 | 56 |
| P11875⁸ | 40 | 58 |
| NP_010628.1⁹ | 30 | 49 |
| AL031853¹⁰ | 31 | 43 |

^{1.}Chinese hamster
^{2.}Human
^{3.}Synechocystis
^{4.}C. elegans
^{5.}Chlamydia sp.
^{6.}Streptomyces sp.
^{7.}Haemophilus
^{8.}E. coli
^{9.}S. cerevisiae
^{10.}S. pombe

### Example 5b

Sequence Analysis of Tagged Embryo-Lethal Line #2092 From the Insertional Mutant Collection

Analysis of Arabidopsis thaliana genomic DNA sequence flanking the right border of the T-DNA insert in line 2092 shows that the T-DNA has inserted into a region of the genome represented by P1 clone MRN17 (GenBank accession AB005243). Further analysis of the insertion site shows that this region contains a gene with sequence identity to genes encoding an alanyl tRNA synthetase.

Using GAP (SeqWeb version 10.0, GCG), pairwise comparisons of the protein sequence (SEQ ID NO:4) and input sequences shown below give a measure of similarity between SEQ ID NO:4 and the indicated sequences, and they are summarized below.

| Genbank Accession # | % Identity | % Similarity |
|---|---|---|
| G2500959¹ | 57.6 | 67.3 |
| AE000353² | 47.3 | 55.3 |
| NP_014980³ | 38.3 | 48.9 |
| AF188718⁴ | 36.9 | 46.3 |
| AB033096⁵ | 34.2 | 42.4 |

^{1.}Synechocystis
^{2.}E. coli
^{3.}yeast
^{4.}Drosophila
^{5.}human

### Example 5c

Sequence Analysis of Tagged Embryo-Lethal Line #7724 From the Insertional Mutant Collection

The sequence of both TAIL PCR border products matches the sequence from the BAC clone F4L23 (Accession AC002387). Further analysis of these products reveals a 20 base pair deletion that occurred upon T-DNA insertion in line #7724, corresponding to base number 60,450 through 60,469, of BAC clone F4L23. Analysis of the DNA sequence from the recovered borders reveals homology to 2′-phosphotransferase genes. Further inspection of recovered border fragments reveals that the T-DNA has inserted in the middle of the coding region for a gene that encodes a protein with greater than 30% identity 2′-phosphotransferase-like genes from microorganisms listed below.

Using GAP (SeqWeb version 10.0, GCG), pairwise comparisons of the protein sequence (SEQ ID NO:6) and input sequences shown below give a measure of similarity between SEQ ID NO:6 and the indicated sequence; and are summarized below.

| Genbank Accession # | % Identity |
|---|---|
| NP_014539¹ | 35.8 |
| CAA22225² | 33.5 |
| CAB16372³ | 33.5 |
| BAA29229⁴ | 32.4 |
| AAB90829⁵ | 30.7 |

^{1.}S. cerevisiae
^{2.}Streptomyces coelicolor
^{3.}S. pombe
^{4.}Pyrococcus horikoshii
^{5.}Archaeoglobus fulgidus

### Example 6a

Isolation and Identification of 1917 cDNA Coding Region

The isolation and characterization of a cDNA clone corresponding to the Arabidopsis thaliana gene encoding arginyl-tRNA synthetase is disclosed in Genbank accession # Z98760.

### Example 6b

Isolation and Identification of 2092 CDNA Coding Region

The full length cDNA for gene 2092 was isolated using the Marathon cDNA amplification kit (CLONETECH). Primers JM99 (5′-ACTTCACTGCCTTCAGAAACCCTTATCACAG-3′: SEQ ID NO:27) and API (part of CLONETECH kit) are used in the first round of amplification on cDNA template generated from 14-day old Arabidopsis seedlings. Then, JM100 (5′-CTTATCACAGGCTTCCCATTCACCAAAAGAC-3′: SEQ ID NO:28) and AP2 (Clonetech) are used in nested PCR reactions to generate the final full-length sequence. Nine independent products are TA cloned, sequenced, and assembled into a single contig using the full sequence of clone 18709 from the Arabidopsis EST project.

### Example 6c

Isolation and Identification of 7724 cDNA Coding Region

Sequence analysis if EST sequences derived from clone 10409 showed that it contained the entire coding region. The two EST sequences derived from the 5′ and 3′ ends of clone 10409 do not overlap. Additional sequencing reactions were performed to complete determination of the sequence of the entire clone. Analysis of the final sequence showed a 2937 bp ORF that encodes the entire deduced protein.

### Example 7a

Expression of Recombinant 1917 Protein in Heterologous Expression Systems

The coding region of the protein, corresponding to the CDNA clone SEQ ID NO:1, is subcloned into previously described expression vectors, and transformed into E. coli using the manufacturer's conditions. Specific examples include plasmids such as pBluescript (Stratagene, La Jolla, Calif.), the pET vector system (Novagen, Inc., Madison, Wis.) pFLAG (International Biotechnologies, Inc., New Haven, Conn.), and pTrcHis (Invitrogen, La Jolla, Calif.). E. coli is cultured, and expression of the 1917 activity is confirmed. Alternatively, eukaryotic expression systems such as cultured insect cells infected with specific viruses may be preferred. Examples of vectors and insect cell lines are described previously. Protein conferring 1917 activity is isolated using standard techniques.

### Example 7b

Expression of Recombinant 2092 Protein in Heterologous Expression Systems

The coding region of the protein, corresponding to the cDNA clone SEQ ID NO:3, is subcloned into previously described expression vectors, and transformed into E. coli using the manufacturer's conditions. Specific examples include plasmids such as pBluescript (Stratagene, La Jolla, Calif.), the pET vector system (Novagen, Inc., Madison, Wis.) pFLAG (International Biotechnologies, Inc., New Haven, Conn.), and pTrcHis (Invitrogen, La Jolla, Calif.). E. coli is cultured, and expression of the 2092 activity is confirmed. Alternatively, eukaryotic expression systems such as cultured insect cells infected with specific viruses may be preferred. Examples of vectors and insect cell lines are described previously. Protein conferring 2092 activity is isolated using standard techniques.

### Example 7c

Expression of Recombinant 7724 Protein in Heterologous Expression Systems

The coding region of the protein, corresponding to the cDNA clone SEQ ID NO:5, is subcloned into previously described expression vectors, and transformed into E. coli using the manufacturer's conditions. Specific examples include plasmids such as pBluescript (Stratagene, La Jolla, Calif.), the pET vector system (Novagen, Inc., Madison, Wis.) pFLAG (International Biotechnologies, Inc., New Haven, Conn.), and pTrcHis (Invitrogen, La Jolla, Calif.). E. coli is cultured, and expression of the 7724 activity is confirmed. Alternatively, eukaryotic expression systems such as cultured insect cells infected with specific viruses may be preferred. Examples of vectors and insect cell lines are described previously. Protein conferring 7724 activity is isolated using standard techniques.

### Example 8a

In vitro Recombination of 1917 Genes by DNA Shuffling

The nucleotide sequence shown in SEQ ID NO:1 is amplified by PCR. The resulting DNA fragment is digested by DNaseI treatment essentially as described (Stemmer et al. (1994) PNAS 91: 10747-10751) and the PCR primers are removed from the reaction mixture. A PCR reaction is carried out without primers and is followed by a PCR reaction with the primers, both as described (Stemmer et al. (1994) PNAS 91: 10747-10751). The resulting DNA fragments are cloned into pTRC99a (Pharmacia, Cat no: 27-5007-01) for use in bacteria, or into pESC vectors (Stratagene Catalog) for use in yeast; and transformed into a bacterial or yeast strain deficient in 1917 activity by electroporation using the Biorad Gene Pulser and the manufacturer's conditions. The transformed bacteria or yeast are grown on medium that contains inhibitory concentrations of an inhibitor of 1917 activity and those colonies that grow in the presence of the inhibitor are selected. Colonies that grow in the presence of normally inhibitory concentrations of inhibitor are picked and purified by repeated restreaking. Their plasmids are purified and the DNA sequences of cDNA inserts from plasmids that pass this test are then determined.

In a similar reaction, PCR-amplified DNA fragments comprising the A. thaliana 1917 gene encoding the protein and PCR-amplified DNA fragments comprising the 1917 gene from E. coli are recombined in vitro and resulting variants with improved tolerance to the inhibitor are recovered as described above.

### Example 8b

In vitro Recombination of 2092 Genes by DNA Shuffling

The nucleotide sequence shown in SEQ ID NO:3 is amplified by PCR. The resulting DNA fragment is digested by DNase I treatment essentially as described (Stemmer et al. (1994) PNAS 91: 10747-10751) and the PCR primers are removed from the reaction mixture. A PCR reaction is carried out without primers and is followed by a PCR reaction with the primers, both as described (Stemmer et al. (1994) PNAS 91: 10747-10751). The resulting DNA fragments are cloned into pTRC99a (Pharmacia, Cat no: 27-5007-01) for use in bacteria, or into pESC vectors (Stratagene Catalog) for use in yeast; and transformed into a bacterial or yeast strain deficient in 2092 activity by electroporation using the Biorad Gene Pulser and the manufacturer's conditions. The transformed bacteria or yeast are grown on medium that contains inhibitory concentrations of an inhibitor of 2092 activity and those colonies that grow in the presence of the inhibitor are selected. Colonies that grow in the presence of normally inhibitory concentrations of inhibitor are picked and purified by repeated restreaking. Their plasmids are purified and the DNA sequences of cDNA inserts from plasmids that pass this test are then determined.

In a similar reaction, PCR-amplified DNA fragments comprising the A. thaliana 2092 gene encoding the protein and PCR-amplified DNA fragments comprising the 2092 gene from E. coli are recombined in vitro and resulting variants with improved tolerance to the inhibitor are recovered as described above.

### Example 8c

In vitro Recombination of 7724 Genes by DNA Shuffling

The nucleotide sequence shown in SEQ ID NO:5 is amplified by PCR. The resulting DNA fragment is digested by DNase I treatment essentially as described (Stemmer et al. (1994) PNAS 91: 10747-10751) and the PCR primers are removed from the reaction mixture. A PCR reaction is carried out without primers and is followed by a PCR reaction with the primers, both as described (Stemmer et al. (1994) PNAS 91: 10747-10751). The resulting DNA fragments are cloned into pTRC99a (Pharmacia, Cat no: 27-5007-01) for use in bacteria, or into pESC vectors (Stratagene Catalog) for use in yeast; and transformed into a bacterial or yeast strain deficient in 7724 activity by electroporation using the Biorad Gene Pulser and the manufacturer's conditions. The transformed bacteria or yeast are grown on medium that contains inhibitory concentrations of an inhibitor of 7724 activity and those colonies that grow in the presence of the inhibitor are selected. Colonies that grow in the presence of normally inhibitory concentrations of inhibitor are picked and purified by repeated restreaking. Their plasmids are purified and the DNA sequences of cDNA inserts from plasmids that pass this test are then determined.

In a similar reaction, PCR-amplified DNA fragments comprising the A. thaliana 7724 gene encoding the protein and PCR-amplified DNA fragments comprising the 7724 gene from E. coli are recombined in vitro and resulting variants with improved tolerance to the inhibitor are recovered as described above.

### Example 9a

In vitro Recombination of 1917 Genes by Staggered Extension Process

The Arabidopsis thaliana 1917 gene encoding the 1917 protein and the E. coli 1917 homologous gene are each cloned into the polylinker of a pBluescript vector. A PCR reaction is carried out essentially as described (Zhao et al. (1998) Nature Biotechnology 16: 258-261) using the “reverse primer” and the “M13-20 primer” (Stratagene Catalog). Amplified PCR fragments are digested with appropriate restriction enzymes and cloned into pTRC99a and mutated 1917 genes are screened as described in Example 8a.

### Example 9b

In vitro Recombination of 2092 Genes by Staggered Extension Process

The Arabidopsis thaliana 2092 gene encoding the 2092 protein and the E. coli 2092 homologous gene are each cloned into the polylinker of a pBluescript vector. A PCR reaction is carried out essentially as described (Zhao et al. (1998) Nature Biotechnology 16: 258-261) using the “reverse primer” and the “M13-20 primer” (Stratagene Catalog). Amplified PCR fragments are digested with appropriate restriction enzymes and cloned into pTRC99a and mutated 2092 genes are screened as described in Example 8b.

### Example 9c

In vitro Recombination of 7724 Genes by Staggered Extension Process

The Arabidopsis thaliana 7724 gene encoding the 7724 protein and the E. coli 7724 homologous gene are each cloned into the polylinker of a pBluescript vector. A PCR reaction is carried out essentially as described (Zhao et al. (1998) Nature Biotechnology 16: 258-261) using the “reverse primer” and the “M13-20 primer” (Stratagene Catalog). Amplified PCR fragments are digested with appropriate restriction enzymes and cloned into pTRC99a and mutated 7724 genes are screened as described in Example 8c.

### Example 10

In vitro Binding Assays

Recombinant 1917, 2092, or 7724 protein is obtained, for example, according to Example 7a, 7b, or 7c, respectively. The protein is immobilized on chips appropriate for ligand binding assays using techniques that are well known in the art. The protein immobilized on the chip is exposed to sample compound in solution according to methods well know in the art. While the sample compound is in contact with the immobilized protein measurements capable of detecting protein-ligand interactions are conducted. Examples of such measurements are SELDI, biacore and FCS, described above. Compounds found to bind the protein are readily discovered in this fashion and are subjected to further characterization.

### Example 11

Plastid Transformation

Transformation Vectors

For expression of a nucleotide sequence encoding a polypeptide having 1917, 2092, or 7724 activity encoding in plant plastids, plastid transformation vector pPH143 or pPH145 (WO 97/32011) is used; and this reference is incorporated herein by reference. The nucleotide sequence is inserted into pPH143 thereby replacing the PROTOX coding sequence. This vector is then used for plastid transformation and selection of transformants for spectinomycin resistance. Alternatively, the nucleotide sequence is inserted in pPH143 so that it replaces the aadH gene. In this case, transformants are selected for resistance to PROTOX inhibitors.

Plastid Transformation

Seeds of Nicotiana tabacum c.v. ‘Xanthi nc’ are germinated seven per plate in a 1″ circular array on T agar medium and bombarded 12-14 days after sowing with 1 μm tungsten particles (M10, Biorad, Hercules, Calif.) coated with DNA from plasmids pPH143 and pPH145 essentially as described (Svab, Z. and Maliga, P. (1993) Proc. Natl. Acad. Sci. USA 90, 913-917). Bombarded seedlings are incubated on T medium for two days after which leaves are excised and placed abaxial side up in bright light (350-500 μmol photons/m²/s) on plates of RMOP medium (Svab, Z., Hajdukiewicz, P. and Maliga, P. (1990) Proc. Natl. Acad. Sci. USA 87, 8526-8530) containing 500 μg/ml spectinomycin dihydrochloride (Sigma, St. Louis, Mo.). Resistant shoots appearing underneath the bleached leaves three to eight weeks after bombardment are subcloned onto the same selective medium, allowed to form callus, and secondary shoots isolated and subcloned. Complete segregation of transformed plastid genome copies (homoplasmicity) in independent subclones is assessed by standard techniques of Southern blotting (Sambrook et al., (1989) Molecular Cloning: A Laboratory Manual, Cold Spring Harbor Laboratory, Cold Spring Harbor). Homoplasmic shoots are rooted aseptically on spectinomycin-containing MS/IBA medium (McBride, K. E. et al. (1994) Proc. Natl. Acad. Sci. USA 91, 7301-7305) and transferred to the greenhouse.

### Example 12a

In vitro assay for Arginyl tRNA Synthetase

The arginyl tRNA synthetase activity assay is derived from Pope et al. (1998) J. Biol. Chem. 273, 31691-31701 and references cited therein. The reaction volumes are preferably the ones described below, but can be varied depending on the experimental requirements. The assay can be performed using 0.2-5 nM, but preferably 1 nM, of an enzyme having arginyl tRNA synthetase activity, 0.1-10 μM, but preferably 1 μM, L-[U-¹⁴C] arginine, and 0.1-10 μM, but preferably 1 μM, of tRNA^{Arg} are mixed in a final volume of 50 μL 50 mM Tris-HCl (pH 7.0-9.0, but preferably 7.9), 1-20 mM, but preferably, 10 mM MgCl₂, 1-100 mM, but preferably 50 mM KCl, and 0.1-20 mM, but preferably 2 mM dithiothreitol. After a time interval, 100 μL of 7% trichloroacetic acid and incubated on ice for 10 minutes. Trichloroacetic acid-precipitate material can be harvested using 0.45 mm polyvinylidene difluoride multiwell plates and counted by scintillation.

### Example 12b

In vitro assay for Alanyl tRNA Synthetase

The alanyl tRNA synthetase activity assay is derived from Pope et al. (1998) J. Biol. Chem. 273, 31691-31701 and references cited therein. The reaction volumes are preferably the ones described below, but can be varied depending on the experimental requirements. The assay can be performed using 0.2-5 nM, but preferably 1 nM, of an enzyme having alanyl tRNA synthetase activity, 0.1-10 μM, but preferably 1 μM, L-[U-¹⁴C] alanine, and 0.1-10 μM, but preferably 1 μM, of tRNA^{Ala} are mixed in a final volume of 50 μL 50 mM Tris-HCl (pH 7.0-9.0, but preferably 7.9), 1-20 mM, but preferably, 10 mM MgCl₂, 1-100 mM, but preferably 50 mM KCl, and 0.1-20 mM, but preferably 2 mM dithiothreitol. After a time interval, 100 μL of 7% trichloroacetic acid and incubated on ice for 10 minutes. Trichloroacetic acid-precipitable material can be harvested using 0.45 mm polyvinylidene difluoride multiwell plates and counted by scintillation.

### Example 12c

In vitro assay for 2′-Phosphotransferase

Many eukaryotes, including the yeast Saccharomyces cerevisiae, humans, and plants contain tRNA gene families whose members contain intervening sequences (Culbertson, M. R. and M. Winey (1989) Yeast 5: 405-427). Joining of the tRNA exons involves a ligase that generates a mature sized tRNA bearing a splice junction 2′-phosphate (Greer et al (1983) Cell 32: 537-546). The removal of the splice junction 2′-phosphate is catalyzed by a 2′phosphotransferase that transfers the splice junction phosphate to NAD, forming ADP-ribose 1′-2′ cyclic phosphate (Culver et al (1993) Science 261: 206-208).

An assay for the 2′phosphotransferase may be performed in which a ligated tRNA with a ³³P- or ³²P-labeled splice junction 2′-phosphate is prepared by in vitro endonucleolytic cleavage and ligation of an (α-³³P) or (α-³²P) ATP-labeled pre-tRNA transcript (McCraith et al (1991) J. Biol. Chem. 266: 11986-11992). The labeled pre-tRNA transcript can be derived by in vitro transcription of a plasmid-borne copy of the end-matured pre-tRNA gene (Reyes et al (1987) Anal. Biochem. 166: 90-106). Alternatively, the pre-tRNA may be synthesized by chemical coupling of the ribonucleic acid building blocks using an oligonucleotide synthesizer. The ligated tRNA with a labeled splice junction may be attached to a scintillant-coated solid support such as a bead, e.g., an SPA bead (Amersham Pharmacia), or a microtiter plate surface, e.g., the Flash Plate (NEN), by covalent attachment or through ligand-ligand interaction, such as biotin-avidin. The radiation given off by the surface-bound, labeled pre-tRNA collides with the scintillator molecules on the solid support. The energy is converted into photons that are measured and quantified by appropriate light-measuring instrumentation. A reaction mixture consisting of an enzyme having 2′-phosphotransferase activity and NAD in a buffer appropriate for the activity of the 2′-phosphotransferase is added to a microtiter plate containing the surface-bound, labeled pre-tRNA. The action of the enzyme will result in the release of the radioisotope from the surface-bound pre-tRNA and, therefore, a decrease in signal. Aspiration and washing steps may be required to eliminate interference from unbound radiolabel.

Alternatively, an oligonucleotide complementary to the labeled pre-tRNA transcript is attached to a solid support. In this case, a reaction mixture consisting of 2′-phosphotransferase, NAD, and unbound, labeled pre-tRNA are incubated for an appropriate period of time and then added to the plate containing the bound, complementary oligonucleotide. The pre-tRNA anneals to the complementary oligonucleotide. The signal arising from any radiolabel remaining on the pre-tRNA is quantified as described above. Aspiration and washing steps may be required to eliminate interference from unbound radiolabel.

The above-disclosed embodiments are illustrative. This disclosure of the invention will place one skilled in the art in possession of many variations of the invention. All such obvious and foreseeable variations are intended to be encompassed by the appended claims.

What is claimed is:

1. A method of identifying herbicidal compounds, comprising:
a) combining a polypeptide comprising an amino acid sequence at least 85% identical to SEQ ID NO:2, 4, or 6 and a compound to be tested for the ability to bind to said polypeptide, under conditions conducive to binding;
b) selecting a compound identified in (a) that binds to said polypeptide;
c) applying a compound selected in (b) to a plant to test for herbicidal activity; and
d) selecting a compound identified in (c) that has herbicidal activity.

2. The method of claim 1, wherein said polypeptide comprises an amino acid sequence at least 95% identical to SEQ ID NO:2, 4, or 6.

3. The method of claim 1, wherein said polypeptide comprises an amino acid sequence at least 99% identical to SEQ ID NO:2, 4, or 6.

4. The method of claim 1, wherein said polypeptide comprises SEQ ID NO:2, 4, or 6.

5. A method of identifying herbicidal compounds, comprising:
a) combining a polypeptide comprising an amino acid sequence at least 85% identical to SEQ ID NO:2, 4, or 6 and a compound to be tested for the ability to inhibit said polypeptide, under conditions conducive to inhibition;
b) selecting a compound identified in (a) that inhibits said polypeptide;
c) applying a compound selected in (b) to a plant to test for herbicidal activity; and
d) selecting a compound identified in (c) that has herbicidal activity.

6. The method of claim 1, wherein said polypeptide comprises an amino acid sequence at least 95% identical to SEQ ID NO:2, 4, or 6.

7. The method of claim 1, wherein said polypeptide comprises an amino acid sequence at least 99% identical to SEQ ID NO:2, 4, or 6.

8. The method of claim 1, wherein said polypeptide comprises SEQ ID NO:2, 4, or 6.


SEQUENCE LISTING (27 sequences)

SEQ ID NO: 1 - DNA, 1773 bp, Arabidopsis thaliana
  CDS (1)..(1773)
atggcagctaatgaagaatttacgggaaatctgaaacgtcaactcgcgaagctctttgatgtttctctaaaattaacggttcctgatgaacctagtgttgagcccttggtggctgcctccgctcttggaaaatttggagattaccaatgtaacaacgcaatgggactatggtccataattaaaggaaagggtactcagttcaagggtcctccagctgttggacaggcccttgttaagagtctccctacttctgagatggtagaatcatgctctgtagctggacctggctttattaatgttgtactatcagctaagtggatggctaagagtattgaaaatatgctcatcgatggagttgacacatgggcacctactctttcggttaagagagctgtagttgatttttcctctcccaacattgcaaaagaaatgcatgttggtcatctaagatcaactatcattggtgacactctagctcgcatgctcgagtactcacatgttgaagttctacgcagaaaccatgttggtgactggggaacacagtttggcatgctaattgagtacctctttgagaaatttcctgatacagatagtgtgaccgagacagcaattggagatcttcaggtgttttacaaggcatcaaaacataaatttgatctggacgaggcctttaaggaaaaagcacaacaggctgtggtccgtctacagggtggtgatcctgtttaccgtaaggcttgggctaagatctgtgacatcagccgaactgagtttgccaaggtttaccaacgccttcgagttgagcttgaagaaaagggagaaagcttttacaaccctcatattgctaaagtaattgaggaattgaatagcaaggggttggttgaagaaagtgaaggtgctcgtgtgattttccttgaaggcttcgacatcccactcatggttgtaaagagtgatggtggttttaactatgcctcaacagatctgactgctctttggtaccggctcaatgaagagaaagctgagtggatcatatatgtgaccgatgttggccagcagcagcactttaatatgttcttcaaagctgccagaaaagcaggttggcttccagacaatgataaaacttaccctagagttaaccatgttggttttggtctcgtccttggggaagatggcaagcgatttagaactcgggcaacagatgtagtccgcctagttgatttgctagatgaggccaagactcgcagtaaacttgcccttattgagcgcggtaaggacaaagaatggacaccggaagaactggaccaaacagctgaggcagttggatatggtgcggtcaagtatgctgacctgaagaacaacagattaacaaattatactttcagctttgatcaaatgcttaatgacaagggaaatacagccgtttaccttctttacgcccatgctcggatctgttcaatcatcagaaagtctggcaaagacatagatgagctgaaaaagacaggaaaattagcattggatcatgcagatgaacgagcactggggcttcacttgcttcgatttgctgagacggtggaggaagcttgtaccaacttattaccgagtgttctgtgcgagtacctctacaatttatctgaacactttaccagattctactccaattgtcaggtcaatggttcaccagaggagacaagccgtctcctactttgtgaagcaacggccatagtcatgcggaaatgcttccaccttcttggaatcactccggtttacaagatttga

SEQ ID NO: 2 - protein, 590 aa, Arabidopsis thaliana
MAANEEFTGNLKRQLAKLFDVSLKLTVPDEPSVEPLVAASALGKFGDYQCNNAMGLWSIIKGKGTQFKGPPAVGQALVKSLPTSEMVESCSVAGPGFINVVLSAKWMAKSIENMLIDGVDTWAPTLSVKRAVVDFSSPNIAKEMHVGHLRSTIIGDTLARMLEYSHVEVLRRNHVGDWGTQFGMLIEYLFEKFPDTDSVTETAIGDLQVFYKASKHKFDLDEAFKEKAQQAVVRLQGGDPVYRKAWAKICDISRTEFAKVYQRLRVELEEKGESFYNPHIAKVIEELNSKGLVEESEGARVIFLEGFDIPLMVVKSDGGFNYASTDLTALWYRLNEEKAEWIIYVTDVGQQQHFNMFFKAARKAGWLPDNDKTYPRVNHVGFGLVLGEDGKRFRTRATDVVRLVDLLDEAKTRSKLALIERGKDKEWTPEELDQTAEAVGYGAVKYADLKNNRLTNYTFSFDQMLNDKGNTAVYLLYAHARICSIIRKSGKDIDELKKTGKLALDHADERALGLHLLRFAETVEEACTNLLPSVLCEYLYNLSEHFTRFYSNCQVNGSPEETSRLLLCEATAIVMRKCFHLLGITPVYKI

SEQ ID NO: 3 - DNA, 2937 bp, Arabidopsis thaliana
  CDS (1)..(2937)
atgaatttctccagagtaaacctcttcgattttcctcttagaccaattttgctttcgcatccttcttctattttcgtttctacacgttttgttaccagaacctctgcaggtgtttctccttctatcttacttcccagatcaactcagtctcctcagattattgctaagagctcatcagtatcagtacagccagtgtctgaggatgctaaggaggattatcagtccaaagatgttagtggagattcaatacggcggcgttttcttgaattctttgcttctcgtggtcataaggtgcttccaagttcgtctcttgtaccagaagatcctaccgtcttgctaacaattgcaggaatgcttcagtttaagcctattttccttggaaaggtacctagagaggttccttgtgcaaccactgcgcaaaggtgtatacgtacgaatgatttggagaatgttgggaaaacggctaggcaccatactttctttgagatgcttgggaactttagctttggtgattacttcaagaaagaagcgataaaatgggcatgggagctttcaactattgagtttgggctaccagctaatagagtttgggttagtatatatgaagacgatgatgaagcttttgaaatctggaagaatgaagttggtgtttctgttgagcggataaagagaatgggtgaagctgacaacttttggactagtggaccaactggtccttgtggtccatgctctgagttgtactatgacttctatcctgagagaggttatgatgaagatgttgatcttggggatgataccagatttattgagttctataatttggttttcatgcagtataacaagacggaagatggattgcttgagcccttgaaacagaagaatatagatactggtcttggtttggaacgtatagctcaaatccttcagaaggttccaaacaactacgagacagatttgatatatccaatcattgcaaagatctcagagttggcgaatatctcatatgactctgcaaatgacaaggcaaagacaagtttaaaagtgattgcagatcacatgcgggcagttgtctatctcatatcagatggtgtttctccttcaaatattggcagaggttatgtggttaggaggctaataagaagagcagttcggaaggggaagtctctcggaataaatggggatatgaatggtaatctaaagggagcgtttttgccagcggttgctgaaaaggtgatagagttgagcacttatattgattcagatgtaaaactaaaggcctcacgcatcattgaggagattaggcaagaagaacttcactttaagaaaactctggaaagaggagaaaagttacttgaccaaaagcttaacgatgcattgtcaattgctgataaaactaaggatacgccttatctggatggaaaagatgcgtttcttctttatgacacatttggctttcctgtggagataactgcagaagttgctgaagaacgtggagtcagtatagatatgaatggttttgaagtggaaatggagaatcaaagacgtcaatctcaagctgctcacaatgttgtaaaactgacagttgaagacgatgctgacatgacgaaaaatattgcagacactgagttccttggatatgacagtctctctgctcgtgctgttgtgaaaagtcttttggtgaatgggaagcctgtgataagggtttctgaaggcagtgaagtagaggttctgctggacagaactccgttctatgctgaatcaggaggtcaaattgcagatcatggttttctttatgttagcagtgatgggaaccaagagaaagctgttgttgaggtaagtgatgtgcagaagtctcttaaaatttttgttcacaagggcactgtaaaaagtggagctctagaagttggcaaggaggtggaagcagcagtagatgcagacttgaggcaacgagcgaaggttcaccatacggccactcatttgctccaatcggcacttaaaaaagtagtaggacaagaaacatcacaggctggttcattagtagcttttgaccgcctcagattcgatttcaattttaatcggtccctgcatgataatgagcttgaggaaatcgaatgcctgatcaataggtggattggggatgctacacgtcttgaaacaaaagtccttcctcttgctgatgcaaaacgtgctggagccatcgcaatgtttggggaaaaatatgatgaaaacgaggttcgtgtagtagaagttcctggtgtctccatggaactttgtggtggcactcatgttggcaatactgcagaaatacgagccttcaagattatctcagaacagggcattgcatctggaatccggcgtatagaagcggttgcaggtgaagcattcattgaatacataaactcacgggattctcaaatgacacgtctatgctcgactctcaaggtgaaagcagaggatgttacaaacagagtggagaatcttctagaggaactacgtgctgctagaaaagaagcctccgacttgcgttcaaaagcagctgtctataaagcatctgtcatatcgaacaaagcatttactgtaggaacttcacagactataagagtgctcgttgagtcgatggatgacaccgatgctgactcattaaagagtgcagctgagcatttgataagcacattggaagatccagtcgctgtggtactaggatcatctccagaaaaagacaaggttagtttagttgctgcatttagtcctggagtagtctccctaggtgttcaagcagggaaattcattggccccatagctaagctgtgtggcggaggaggtggtggaaagcccaattttgctcaggcaggcggcagaaagcctgaaaatctcccaagtgccttagagaaagctcgggaagatctcgtggcaactctattcgaaaagctagggtga

SEQ ID NO: 4 - protein, 978 aa, Arabidopsis thaliana
MNFSRVNLFDFPLRPILLSHPSSIFVSTRFVTRTSAGVSPSILLPRSTQSPQIIAKSSSVSVQPVSEDAKEDYQSKDVSGDSIRRRFLEFFASRGHKVLPSSSLVPEDPTVLLTIAGMLQFKPIFLGKVPREVPCATTAQRCIRTNDLENVGKTARHHTFFEMLGNFSFGDYFKKEAIKWAWELSTIEFGLPANRVWVSIYEDDDEAFEIWKNEVGVSVERIKRMGEADNFWTSGPTGPCGPCSELYYDFYPERGYDEDVDLGDDTRFIEFYNLVFMQYNKTEDGLLEPLKQKNIDTGLGLERIAQILQKVPNNYETDLIYPIIAKISELANISYDSANDKAKTSLKVIADHMRAVVYLISDGVSPSNIGRGYVVRRLIRRAVRKGKSLGINGDMNGNLKGAFLPAVAEKVIELSTYIDSDVKLKASRIIEEIRQEELHFKKTLERGEKLLDQKLNDALSIADKTKDTPYLDGKDAFLLYDTFGFPVEITAEVAEERGVSIDMNGFEVEMENQRRQSQAAHNVVKLTVEDDADMTKNIADTEFLGYDSLSARAVVKSLLVNGKPVIRVSEGSEVEVLLDRTPFYAESGGQIADHGFLYVSSDGNQEKAVVEVSDVQKSLKIFVHKGTVKSGALEVGKEVEAAVDADLRQRAKVHHTATHLLQSALKKVVGQETSQAGSLVAFDRLRFDFNFNRSLHDNELEEIECLINRWIGDATRLETKVLPLADAKRAGAIAMFGEKYDENEVRVVEVPGVSMELCGGTHVGNTAEIRAFKIISEQGIASGIRRIEAVAGEAFIEYINSRDSQMTRLCSTLKVKAEDVTNRVENLLEELRAARKEASDLRSKAAVYKASVISNKAFTVGTSQTIRVLVESMDDTDADSLKSAAEHLISTLEDPVAVVLGSSPEKDKVSLVAAFSPGVVSLGVQAGKFIGPIAKLCGGGGGGKPNFAQAGGRKPENLPSALEKAREDLVATLFEKLG

SEQ ID NO: 5 - DNA, 774 bp, Arabidopsis thaliana
  CDS (1)..(774)
atggatgcttcaaatcccaattcttctagaaaatctaatgtctcttccttcgctcagtccagtcgaagcggtggtagaggaggaggatatgagagagataacgatcgacggagacctcagggtcgtggcgacggtggaggcggaaaggatagaatcgatgcacttggacgactcttgacgagaatattgcgacatatggctactgagctgagattgaacatgagaggtgatggttttgttaaagttgaagatttacttaacctgaatttgaaaacttctgcaaatattcagttaaagtcacacacgattgatgaaattagagaggctgtgagaagggacaataagcaacggtttagtctcatcgatgagaatggagagctcttgattcgcgctaaccaaggccattcgatcacgacggttgagtcagagaagttacttaaaccaatactgtcaccagaagaagctccagtgtgtgtacatggaacttataggaagaatttggaatccatcttagcatcgggcttaaagcgtatgaatagaatgcatgttcacttctcttgtggattaccaacagatggtgaagtgattagtggcatgagaagaaatgtaaatgttatcatcttcctcgacatcaagaaagctcttgaagatgggattgcgttctacatatcagacaacaaagtgattttgactgaaggcattgatggtgtattgcctgtcgattacttccagaagatcgagtcttggcctgatcggcaatccatacctttctga

SEQ ID NO: 6 - protein, 257 aa, Arabidopsis thaliana
MDASNPNSSRKSNVSSFAQSSRSGGRGGGYERDNDRRRPQGRGDGGGGKDRIDALGRLLTRILRHMATELRLNMRGDGFVKVEDLLNLNLKTSANIQLKSHTIDEIREAVRRDNKQRFSLIDENGELLIRANQGHSITTVESEKLLKPILSPEEAPVCVHGTYRKNLESILASGLKRMNRMHVHFSCGLPTDGEVISGMRRNVNVIIFLDIKKALEDGIAFYISDNKVILTEGIDGVLPVDYFQKIESWPDRQSIPF

SEQ ID NO: 7 - DNA, 3138 bp, Arabidopsis thaliana
  CDS (17)..(2953)
ctcctcatactctctgatgaatttctccagagtaaacctcttcgattttcctcttagaccaattttgctttcgcatccttcttctattttcgtttctacacgttttgttaccagaacctctgcaggtgtttctccttctatcttacttcccagatcaactcagtctcctcagattattgctaagagctcatcagtatcagtacagccagtgtctgaggatgctaaggaggattatcagtccaaagatgttagtggagattcaatacggcggcgttttcttgaattctttgcttctcgtggtcataaggtgcttccaagttcgtctcttgtaccagaagatcctaccgtcttgctaacaattgcaggaatgcttcagtttaagcctattttccttggaaaggtacctagagaggttccttgtgcaaccactgcgcaaaggtgtatacgtacgaatgatttggagaatgttgggaaaacggctaggcaccatactttctttgagatgcttgggaactttagctttggtgattacttcaagaaagaagcgataaaatgggcatgggagctttcaactattgagtttgggctaccagctaatagagtttgggttagtatatatgaagacgatgatgaagcttttgaaatctggaagaatgaagttggtgtttctgttgagcggataaagagaatgggtgaagctgacaacttttggactagtggaccaactggtccttgtggtccatgctctgagttgtactatgacttctatcctgagagaggttatgatgaagatgttgatcttggggatgataccagatttattgagttctataatttggttttcatgcagtataacaagacggaagatggattgcttgagcccttgaaacagaagaatatagatactggtcttggtttggaacgtatagctcaaatccttcagaaggttccaaacaactacgagacagatttgatatatccaatcattgcaaagatctcagagttggcgaatatctcatatgactctgcaaatgacaaggcaaagacaagtttaaaagtgattgcagatcacatgcgggcagttgtctatctcatatcagatggtgtttctccttcaaatattggcagaggttatgtggttaggaggctaataagaagagcagttcggaaggggaagtctctcggaataaatggggatatgaatggtaatctaaagggagcgtttttgccagcggttgctgaaaaggtgatagagttgagcacttatattgattcagatgtaaaactaaaggcctcacgcatcattgaggagattaggcaagaagaacttcactttaagaaaactctggaaagaggagaaaagttacttgaccaaaagcttaacgatgcattgtcaattgctgataaaactaaggatacgccttatctggatggaaaagatgcgtttcttctttatgacacatttggctttcctgtggagataactgcagaagttgctgaagaacgtggagtcagtatagatatgaatggttttgaagtggaaatggagaatcaaagacgtcaatctcaagctgctcacaatgttgtaaaactgacagttgaagacgatgctgacatgacgaaaaatattgcagacactgagttccttggatatgacagtctctctgctcgtgctgttgtgaaaagtcttttggtgaatgggaagcctgtgataagggtttctgaaggcagtgaagtagaggttctgctggacagaactccgttctatgctgaatcaggaggtcaaattgcagatcatggttttctttatgttagcagtgatgggaaccaagagaaagctgttgttgaggtaagtgatgtgcagaagtctcttaaaatttttgttcacaagggcactgtaaaaagtggagctctagaagttggcaaggaggtggaagcagcagtagatgcagacttgaggcaacgagcgaaggttcaccatacggccactcatttgctccaatcggcacttaaaaaagtagtaggacaagaaacatcacaggctggttcattagtagcttttgaccgcctcagattcgatttcaattttaatcggtccctgcatgataatgagcttgaggaaatcgaatgcctgatcaataggtggattggggatgctacacgtcttgaaacaaaagtccttcctcttgctgatgcaaaacgtgctggagccatcgcaatgtttggggaaaaatatgatgaaaacgaggttcgtgtagtagaagttcctggtgtctccatggaactttgtggtggcactcatgttggcaatactgcagaaatacgagccttcaagattatctcagaacagggcattgcatctggaatccggcgtatagaagcggttgcaggtgaagcattcattgaatacataaactcacgggattctcaaatgacacgtctatgctcgactctcaaggtgaaagcagaggatgttacaaacagagtggagaatcttctagaggaactacgtgctgctagaaaagaagcctccgacttgcgttcaaaagcagctgtctataaagcatctgtcatatcgaacaaagcatttactgtaggaacttcacagactataagagtgctcgttgagtcgatggatgacaccgatgctgactcattaaagagtgcagctgagcatttgataagcacattggaagatccagtcgctgtggtactaggatcatctccagaaaaagacaaggttagtttagttgctgcatttagtcctggagtagtctccctaggtgttcaagcagggaaattcattggccccatagctaagctgtgtggcggaggaggtggtggaaagcccaattttgctcaggcaggcggcagaaagcctgaaaatctcccaagtgccttagagaaagctcgggaagatctcgtggcaactctattcgaaaagctagggtgaagcacaaacttcaaaagtgatctgcgtgtacagagagaaggaagagcacattgcttgattctagacaagtgtattgcatgtatagatgatagacattaaagatatttgatgtatctagtttttgaacattaaatgatcaatgacatttcttttaatgaaaaaaaaaaaaaaaaaaaaaaaaaaaa

SEQ ID NO: 8 - protein, 978 aa, Arabidopsis thaliana; identical to SEQ ID NO: 4

SEQ ID NO: 9 - DNA, 16 bp, Artificial Sequence
  Description of Artificial Sequence oligonucleotide
ngtcgaswganawgaa

SEQ ID NO: 10 - DNA, 16 bp, Artificial Sequence
  Description of Artificial Sequence oligonucleotide
tgwgnagsancasaga

SEQ ID NO: 11 - DNA, 16 bp, Artificial Sequence
  Description of Artificial Sequence oligonucleotide
agwgnagwancawagg

SEQ ID NO: 12 - DNA, 16 bp, Artificial Sequence
  Description of Artificial Sequence oligonucleotide
sttgntastnctntgc

SEQ ID NO: 13 - DNA, 15 bp, Artificial Sequence
  Description of Artificial Sequence oligonucleotide
ntcgastwtsgwgtt

SEQ ID NO: 14 - DNA, 16 bp, Artificial Sequence
  Description of Artificial Sequence oligonucleotide
wgtgnagwancanaga

SEQ ID NO: 15 - DNA, 29 bp, Artificial Sequence
  Description of Artificial Sequence oligonucleotide
attaggcaccccaggctttacactttatg

SEQ ID NO: 16 - DNA, 30 bp, Artificial Sequence
  Description of Artificial Sequence oligonucleotide
gtatgttgtgtggaattgtgagcggataac

SEQ ID NO: 17 - DNA, 30 bp, Artificial Sequence
  Description of Artificial Sequence oligonucleotide
taacaatttcacacaggaaacagctatgac

SEQ ID NO: 18 - DNA, 34 bp, Artificial Sequence
  Description of Artificial Sequence oligonucleotide
tagcatctgaatttcataaccaatctcgatacac

SEQ ID NO: 19 - DNA, 34 bp, Artificial Sequence
  Description of Artificial Sequence oligonucleotide
gcttcctattatatcttcccaaattaccaataca

SEQ ID NO: 20 - DNA, 34 bp, Artificial Sequence
  Description of Artificial Sequence oligonucleotide
gccttttcagaaatggataaatagccttgcttcc

SEQ ID NO: 21 - DNA, 1030 bp, Arabidopsis thaliana
  CDS (74)..(847)
tcgacttcctcttcctctgactttgagcagctctgtcttcttctcgaaatcgtctcctgtttcttctgctttcatggatgcttcaaatcccaattcttctagaaaatctaatgtctcttccttcgctcagtccagtcgaagcggtggtagaggaggaggatatgagagagataacgatcgacggagacctcagggtcgtggcgacggtggaggcggaaaggatagaatcgatgcacttggacgactcttgacgagaatattgcgacatatggctactgagctgagattgaacatgagaggtgatggttttgttaaagttgaagatttacttaacctgaatttgaaaacttctgcaaatattcagttaaagtcacacacgattgatgaaattagagaggctgtgagaagggacaataagcaacggtttagtctcatcgatgagaatggagagctcttgattcgcgctaaccaaggccattcgatcacgacggttgagtcagagaagttacttaaaccaatactgtcaccagaagaagctccagtgtgtgtacatggaacttataggaagaatttggaatccatcttagcatcgggcttaaagcgtatgaatagaatgcatgttcacttctcttgtggattaccaacagatggtgaagtgattagtggcatgagaagaaatgtaaatgttatcatcttcctcgacatcaagaaagctcttgaagatgggattgcgttctacatatcagacaacaaagtgattttgactgaaggcattgatggtgtattgcctgtcgattacttccagaagatcgagtcttggcctgatcggcaatccatacctttctgattcatataattcaacatcatgcgaagattgacaggatcctatgacaatgattgtgaggattcttctgaaccttgattatgtaatgttgtctcagtgttttcaattgcacatatgacaatttatgaaaactttcaagattatgttgtttcctttgcccaaagaaaaaaaaaaaaaaaaaaaaaa

SEQ ID NO: 22 - protein, 257 aa, Arabidopsis thaliana; identical to SEQ ID NO: 6

SEQ ID NO: 23 - DNA, 1929 bp, Arabidopsis thaliana
  CDS (1)..(1929)
atgttcattttcccaaaagacgaaaacagaagagaaactttaacgacaaagctccgtttctccgccgatcatctgacttttaccaccgtgacagaaaaattgagagcaacggcttggagatttgctttctcatccagagctaagtccgtggtagcaatggcagctaatgaagaatttacgggaaatctgaaacgtcaactcgcgaagctctttgatgtttctctaaaattaacggttcctgatgaacctagtgttgagcccttggtggctgcctccgctcttggaaaatttggagattaccaatgtaacaacgcaatgggactatggtccataattaaaggaaagggtactcagttcaagggtcctccagctgttggacaggcccttgttaagagtctccctacttctgagatggtagaatcatgctctgtagctggacctggctttattaatgttgtactatcagctaagtggatggctaagagtattgaaaatatgctcatcgatggagttgacacatgggcacctactctttcggttaagagagctgtagttgatttttcctctcccaacattgcaaaagaaatgcatgttggtcatctaagatcaactatcattggtgacactctagctcgcatgctcgagtactcacatgttgaagttctacgcagaaaccatgttggtgactggggaacacagtttggcatgctaattgagtacctctttgagaaatttcctgatacagatagtgtgaccgagacagcaattggagatcttcaggtgttttacaaggcatcaaaacataaatttgatctggacgaggcctttaaggaaaaagcacaacaggctgtggtccgtctacagggtggtgatcctgtttaccgtaaggcttgggctaagatctgtgacatcagccgaactgagtttgccaaggtttaccaacgccttcgagttgagcttgaagaaaagggagaaagcttttacaaccctcatattgctaaagtaattgaggaattgaatagcaaggggttggttgaagaaagtgaaggtgctcgtgtgattttccttgaaggcttcgacatcccactcatggttgtaaagagtgatggtggttttaactatgcctcaacagatctgactgctctttggtaccggctcaatgaagagaaagctgagtggatcatatatgtgaccgatgttggccagcagcagcactttaatatgttcttcaaagctgccagaaaagcaggttggcttccagacaatgataaaacttaccctagagttaaccatgttggttttggtctcgtccttggggaagatggcaagcgatttagaactcgggcaacagatgtagtccgcctagttgatttgctagatgaggccaagactcgcagtaaacttgcccttattgagcgcggtaaggacaaagaatggacaccggaagaactggaccaaacagctgaggcagttggatatggtgcggtcaagtatgctgacctgaagaacaacagattaacaaattatactttcagctttgatcaaatgcttaatgacaagggaaatacagccgtttaccttctttacgcccatgctcggatctgttcaatcatcagaaagtctggcaaagacatagatgagctgaaaaagacaggaaaattagcattggatcatgcagatgaacgagcactggggcttcacttgcttcgatttgctgagacggtggaggaagcttgtaccaacttattaccgagtgttctgtgcgagtacctctacaatttatctgaacactttaccagattctactccaattgtcaggtcaatggttcaccagaggagacaagccgtctcctactttgtgaagcaacggccatagtcatgcggaaatgcttccaccttcttggaatcactccggtttacaagatttga

SEQ ID NO: 24 - protein, 642 aa, Arabidopsis thaliana
MFIFPKDENRRETLTTKLRFSADHLTFTTVTEKLRATAWRFAFSSRAKSVVAMAANEEFTGNLKRQLAKLFDVSLKLTVPDEPSVEPLVAASALGKFGDYQCNNAMGLWSIIKGKGTQFKGPPAVGQALVKSLPTSEMVESCSVAGPGFINVVLSAKWMAKSIENMLIDGVDTWAPTLSVKRAVVDFSSPNIAKEMHVGHLRSTIIGDTLARMLEYSHVEVLRRNHVGDWGTQFGMLIEYLFEKFPDTDSVTETAIGDLQVFYKASKHKFDLDEAFKEKAQQAVVRLQGGDPVYRKAWAKICDISRTEFAKVYQRLRVELEEKGESFYNPHIAKVIEELNSKGLVEESEGARVIFLEGFDIPLMVVKSDGGFNYASTDLTALWYRLNEEKAEWIIYVTDVGQQQHFNMFFKAARKAGWLPDNDKTYPRVNHVGFGLVLGEDGKRFRTRATDVVRLVDLLDEAKTRSKLALIERGKDKEWTPEELDQTAEAVGYGAVKYADLKNNRLTNYTFSFDQMLNDKGNTAVYLLYAHARICSIIRKSGKDIDELKKTGKLALDHADERALGLHLLRFAETVEEACTNLLPSVLCEYLYNLSEHFTRFYSNCQVNGSPEETSRLLLCEATAIVMRKCFHLLGITPVYKI

SEQ ID NO: 25 - DNA, 20 bp, Artificial Sequence
  Description of Artificial Sequence oligonucleotide
gcggacatctacatttttga

SEQ ID NO: 26 - DNA, 31 bp, Artificial Sequence
  Description of Artificial Sequence oligonucleotide
acttcactgccttcagaaacccttatcacag

SEQ ID NO: 27 - DNA, 31 bp, Artificial Sequence
  Description of Artificial Sequence oligonucleotide
cttatcacaggcttcccattcaccaaaagac